SEQUENCE LISTING (8 sequences)

SEQ ID NO: 1 - DNA, 750 bp, Aspergillus clavatus
atggtgtcgttcaagtatcttttcctggcagcctctgccctgggagcgctggctgctcctgttgaagtggaagagtcttcctggttcaacgagacggctctgcacgagtttgctgagcgtgcgggcacccccagctcgaccggatggaataatggctactactattccttctggactgacaatggtggcactgttaactaccagaacggcaacggcggttcgtactcagtccagtggaaggacacaggcaactttgttggcggcaagggctggaaccctggaagcgctcggtatgtctttgtgtatctagagaaagccagagggtgtacaaagaattaaccggcatgcagaaccatcaactactcgggcagcttcaaccccagcggcaatgcctatctcaccgtgtatggctggaccactaatccccttgtggagtactacatagtggagaactatggtacttataaccctggaaatggtggcacctataggggcagtgtatactctgatggtgccaactataacatctacacggccactcgctacaatgctccctccattgagggtgataagaccttcacccagtactggtctgtgcgccagagtaagcggactggcggcacagttacaactgccaaccacttcaacgcctgggctcagctgggtatgagtttgggcacccacaactatcagatcgttgctactgagggctaccagagcagcggttcctcctccattactgtctattaa

SEQ ID NO: 2 - DNA, 690 bp, Aspergillus clavatus
atggtgtcgttcaagtatcttttcctggcagcctctgccctgggagcgctggctgctcctgttgaagtggaagagtcttcctggttcaacgagacggctctgcacgagtttgctgagcgtgcgggcacccccagctcgaccggatggaataatggctactactattccttctggactgacaatggtggcactgttaactaccagaacggcaacggcggttcgtactcagtccagtggaaggacacaggcaactttgttggcggcaagggctggaaccctggaagcgctcgaaccatcaactactcgggcagcttcaaccccagcggcaatgcctatctcaccgtgtatggctggaccactaatccccttgtggagtactacatagtggagaactatggtacttataaccctggaaatggtggcacctataggggcagtgtatactctgatggtgccaactataacatctacacggccactcgctacaatgctccctccattgagggtgataagaccttcacccagtactggtctgtgcgccagagtaagcggactggcggcacagttacaactgccaaccacttcaacgcctgggctcagctgggtatgagtttgggcacccacaactatcagatcgttgctactgagggctaccagagcagcggttcctcctccattactgtctattaa

SEQ ID NO: 3 - DNA, 636 bp, Aspergillus clavatus
gctcctgttgaagtggaagagtcttcctggttcaacgagacggctctgcacgagtttgctgagcgtgcgggcacccccagctcgaccggatggaataatggctactactattccttctggactgacaatggtggcactgttaactaccagaacggcaacggcggttcgtactcagtccagtggaaggacacaggcaactttgttggcggcaagggctggaaccctggaagcgctcgaaccatcaactactcgggcagcttcaaccccagcggcaatgcctatctcaccgtgtatggctggaccactaatccccttgtggagtactacatagtggagaactatggtacttataaccctggaaatggtggcacctataggggcagtgtatactctgatggtgccaactataacatctacacggccactcgctacaatgctccctccattgagggtgataagaccttcacccagtactggtctgtgcgccagagtaagcggactggcggcacagttacaactgccaaccacttcaacgcctgggctcagctgggtatgagtttgggcacccacaactatcagatcgttgctactgagggctaccagagcagcggttcctcctccattactgtctattaa

SEQ ID NO: 4 - DNA, 39 bp, Artificial Sequence
  Primer 1
ccgcggccgcaccatggtgtcgttcaagtatcttttcct

SEQ ID NO: 5 - DNA, 40 bp, Artificial Sequence
  Primer 2
ccggcgcgcccttattaatagacagtaatggaggaggaac

SEQ ID NO: 6 - protein, 229 aa, Aspergillus clavatus
MVSFKYLFLAASALGALAAPVEVEESSWFNETALHEFAERAGTPSSTGWNNGYYYSFWTDNGGTVNYQNGNGGSYSVQWKDTGNFVGGKGWNPGSARTINYSGSFNPSGNAYLTVYGWTTNPLVEYYIVENYGTYNPGNGGTYRGSVYSDGANYNIYTATRYNAPSIEGDKTFTQYWSVRQSKRTGGTVTTANHFNAWAQLGMSLGTHNYQIVATEGYQSSGSSSITVY

SEQ ID NO: 7 - protein, 211 aa, Aspergillus clavatus
APVEVEESSWFNETALHEFAERAGTPSSTGWNNGYYYSFWTDNGGTVNYQNGNGGSYSVQWKDTGNFVGGKGWNPGSARTINYSGSFNPSGNAYLTVYGWTTNPLVEYYIVENYGTYNPGNGGTYRGSVYSDGANYNIYTATRYNAPSIEGDKTFTQYWSVRQSKRTGGTVTTANHFNAWAQLGMSLGTHNYQIVATEGYQSSGSSSITVY

SEQ ID NO: 8 - protein, 189 aa, Aspergillus clavatus
AGTPSSTGWNNGYYYSFWTDNGGTVNYQNGNGGSYSVQWKDTGNFVGGKGWNPGSARTINYSGSFNPSGNAYLTVYGWTTNPLVEYYIVENYGTYNPGNGGTYRGSVYSDGANYNIYTATRYNAPSIEGDKTFTQYWSVRQSKRTGGTVTTANHFNAWAQLGMSLGTHNYQIVATEGYQSSGSSSITVY

(19) United States
(12) Patent Application Publication
(10) Pub. No.: US 2019/0090525 A1
(43) Pub. Date: Mar. 28, 2019

(54) XYLANASES FOR SOLUBILIZING ARABINOXYLAN-CONTAINING MATERIAL

(71) Applicant: DUPONT NUTRITION BIOSCIENCES APS, Copenhagen (DK)

(72) Inventors: Susan Arent Lund, Brabrand (DK); Brian Soegaard Laursen, Kalundborg (DK); Elijah G. Kiarie, Guelph (CA); Luis Fernando Romero Millan, Wilshire (GB); Zhenghong Zhang, Palo Alto, CA (US); Rosalyn Lau, Shanghai (CN); Zheyong Yu, Shanghai (CN); Mads Broegger Pedersen, Frederiksberg (DK); Soeren Dalsgaard, Silkeborg (DK)

(21) Appl. No.: 16/156,572

(22) Filed: Oct. 10, 2018

Related U.S. Application Data
(62) Division of application No. 14/418,905, filed on Jan. 30, 2015, now abandoned.

(30) Foreign Application Priority Data
Aug. 3, 2012 (CN) PCT/CN2012/079657
Jul. 22, 2013 (CN) 201310308689.X

(51) Int. Cl. A23L 29/00; A23K 10/14; C12N 9/24; A23K 20/189; A23L 7/10; A23K 10/38; A23L 33/21; C12C 5/00

(52) U.S. Cl. CPC A23L 29/06; A23K 10/14; C12N 9/2482; A23K 20/189; C12C 5/004; A23L 7/115; A23K 10/38; A23L 33/21; Y02P 60/873

(57) ABSTRACT

The present invention relates to a method of preparing a corn based product said method comprising contacting a plant composition comprising (consisting of or consisting essentially of) corn or a corn by-product or a combination thereof with a xylanase comprising: i) a polypeptide as set forth in SEQ ID No. 8 or SEQ ID No. 7 or SEQ ID No. 6; or ii) a variant, fragment homologue, fragment or derivative thereof having at least 85% identity with SEQ ID No. 8 or SEQ ID No. 7 or SEQ ID No. 6; or with a xylanase encoded by: a) a nucleotide sequence shown herein as SEQ ID No. 3. SEQ ID No. 2 or SEQ ID No. 1; or b) a nucleotide sequence which can hybridize to the complement of SEQ ID No. 3. SEQ ID No. 2 or SEQ ID No. 1 under high stringency conditions: or c) a nucleotide sequence which has at least 80% identity with SEQ ID No. 3. SEQ ID No. 2 or SEQ ID No. 1. The present invention further relates to the use of the enzyme to produce corn based feedstuffs. The present invention yet further relates to a method of producing a fermented beverage comprising the step of contacting a mash and/or a wort with a xylanase comprising a polypeptide as set forth in SEQ ID No. 8 or SEQ ID No. 7 or SEQ ID No. 6; or a variant, fragment, homologue or derivative thereof having at least 85% identity with SEQ ID No. 8 or SEQ ID No. 7 or SEQ ID No. 6; or encoded by a nucleotide sequence shown herein as SEQ ID No. 3, SEQ ID No. 2 or SEQ ID No. 1, or a nucleotide sequence which can hybridize to the complement of SEQ ID No. 3, SEQ ID No. 2 or SEQ ID No. 1 under high stringency conditions, or a nucleotide sequence which has at least 80% identity with SEQ ID No. 3, SEQ ID No. 2 or SEQ ID No. 1.

### FIELD OF THE INVENTION

The present invention relates to the preparation of feed products and/or feed additive compositions which in use may improve the performance of a subject or improve digestibility (e.g. nutrient digestibility) or improve feed efficiency in a subject. In particular, the present invention relates to the use of a xylanase having unexpectedly good activity in the solubilisation of pentosans in corn and corn by-products. The invention further relates to uses of a xylanase in for example animal feed.

### BACKGROUND OF THE INVENTION

Many plants and plant by-products are utilised for feed for animals such as soybean, alfalfa, barley; birdsfoot trefoil; Brassica spp—such as kale, rapeseed, canola, swede and turnip; clover, corn (maize); oats, millet, sorghum, soybean and wheat. For such applications it is advantageous to break down complex carbohydrates derived from plant cell wall material.

Hemicellulose and cellulose found in plant cell walls are potential energy sources, as they consist of C5- and C6-saccharides. C6-saccharides can be used as energy source by the animal, while oligo C5-saccharides can be transformed into short chain fatty acids by the micro flora present in the animal gut (van den Broek et al., 2008 Molecular Nutrition & Food Research, 52, 146-63). Aiding solubilisation of such C5- and C6-saccharides, therefore, allows increased energy utilisation of such plants.

Enzymes, such as xylanases (e.g., endo-β-1,4-xylanases (EC 3.2.1.8)), have been taught to have utility in the breakdown of complex carbohydrates derived from plant cell walls. Xylanase is the name given to a class of enzymes which degrade the linear polysaccharide beta-1,4-xylan into xylooligosaccharides or xylose, thus breaking down hemicellulose, one of the major components of plant cell walls.

For example, Econase® XT is an endo-1,4-β-xylanase from Trichoderma reesei available from ABVista.

It is well known in the art that the functionality of different xylanases (derived from different microorganisms or plants) differs enormously.

Corn and corn by-products are utilised in a number of industries such as in animal feed.

However, there is variance in the structure of hemicellulose and cellulose between different plants which affects the ability of different enzymes to solubilise such saccharides.

For example, Econase® XT can solubilise pentosans in wheat. As shown herein, this commercially available xylanase is not good at the solubilisation of saccharides (e.g. pentosans) in corn or corn by-products.

Accordingly, there is a need for a method of preparing feed and/or feed additive compositions which may result in increased solubilisation of pentosans from corn or corn by-products.

### BRIEF DESCRIPTION OF THE DRAWINGS

FIG. 1 shows a nucleotide sequence (SEQ ID No. 1) encoding the AclXyn5 xylanase. The nucleotides which are in lowercase show the intron sequence. The signal sequence is shown bold (upper case).

FIG. 2 shows a nucleotide sequence (SEQ ID No. 2) encoding the AclXyn5 xylanase of the present invention. The signal sequence is shown bold (upper case).

FIG. 3 shows a nucleotide sequence (SEQ ID No. 3) encoding the AclXyn5 xylanase of the present invention.

FIG. 4 shows a polypeptide sequence (SEQ ID No. 6) of AclXyn5 xylanase of the present invention. This is the pre-protein. The bolded portion of the sequence reflects an N terminal signal peptide which can be cleaved before the enzyme is matured.

FIG. 5 shows a polypeptide sequence (SEQ ID No. 7) of the AclXyn5 xylanase. This is an active form of the enzyme. This may be referred to herein as the mature form of the enzyme.

FIG. 6 shows a polypeptide sequence (SEQ ID No. 8) of the AclXyn5 xylanase. This is also an active form of the enzyme which may arise from posttranslational processing.

FIG. 7 shows the map of plasmid pZZH159.

FIG. 8 shows the pH profile of AclXyn5. AclXyn5 was found to have an optimum pH at about 5, and was found to retain greater than 70% of maximum activity between pH 4.3 and 6.6.

FIG. 9 shows the temperature profile of AclXyn5. AclXyn5 was found to have an optimum temperature of 60° C., and was found to retain greater than 70% of maximum activity between 49° C. and 64° C.

FIG. 10 shows a scheme of an auto-analyzer for the determination of pentosan by an automated phloroglucinol method: (a) Acetic acid mixed with HCl; (b) air bubbling; (c) phloroglucinol in ethanol; (d) sample; (e) sample accelerator; (f) flow cell way-out; (g) peristaltic pump; (h) glass coil; (i) thermostat (96° C.); (j) multiple wavelength spectrophotometer (410, 510, 550, and 620 nm); (k) waste; (l) computer (Rouau & Surget, 1994 Carbohydrate Polymers, 24, 123-32).

FIG. 11 shows solubilisation of pentosans from cDDGS as a function of xylanase dosage. The xylanases used were AclXyn 5 compared with the benchmark xylanase Econase® XT. The order of legends indicates the ranking at the highest xylanase dose (36 mg/kg feed).

FIG. 12 shows pentosan (C-5 sugar) release (solubilisation of pentosans) from respectively corn and cDDGS with and without AclXyn5 xylanase addition (36 mg/kg feed) after 18 h incubation.

FIG. 13 shows the effect of the xylanase and protease treatments alone and in combination on the solubilization of pentosan and protein from insoluble corn DDGS. Letters a-d are significant different according to on-way ANOVA and Holm-Sidak comparisons with overall significance level at P=0.05. Error bars indicate S.D.

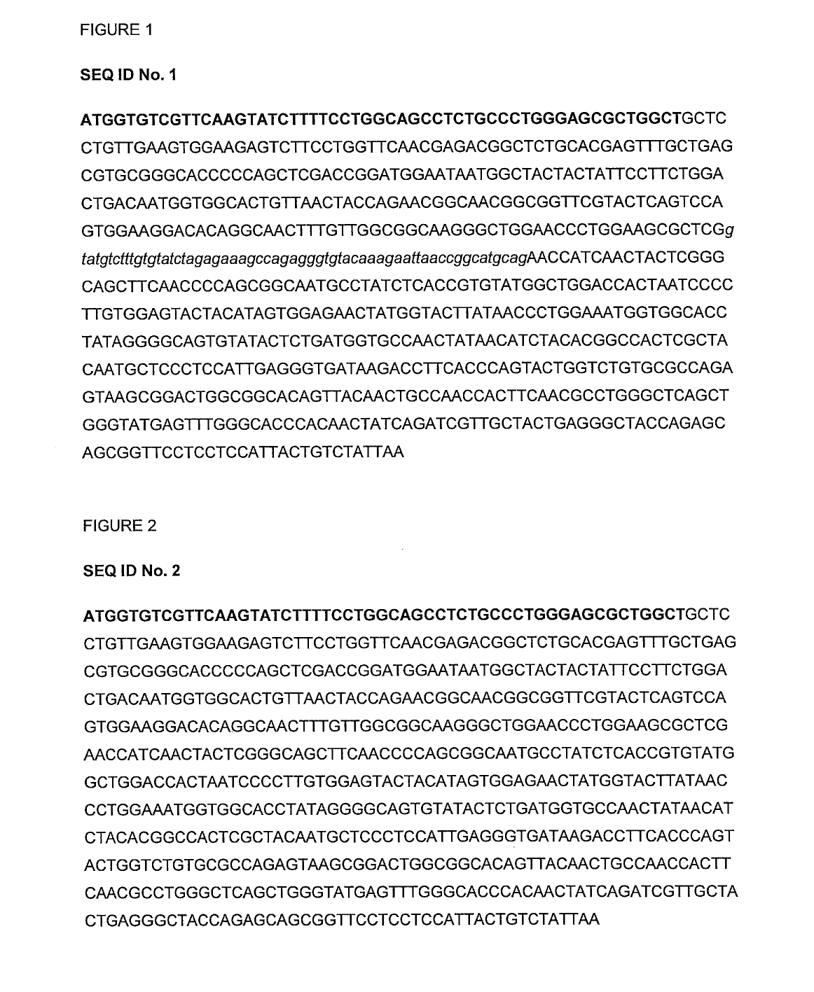

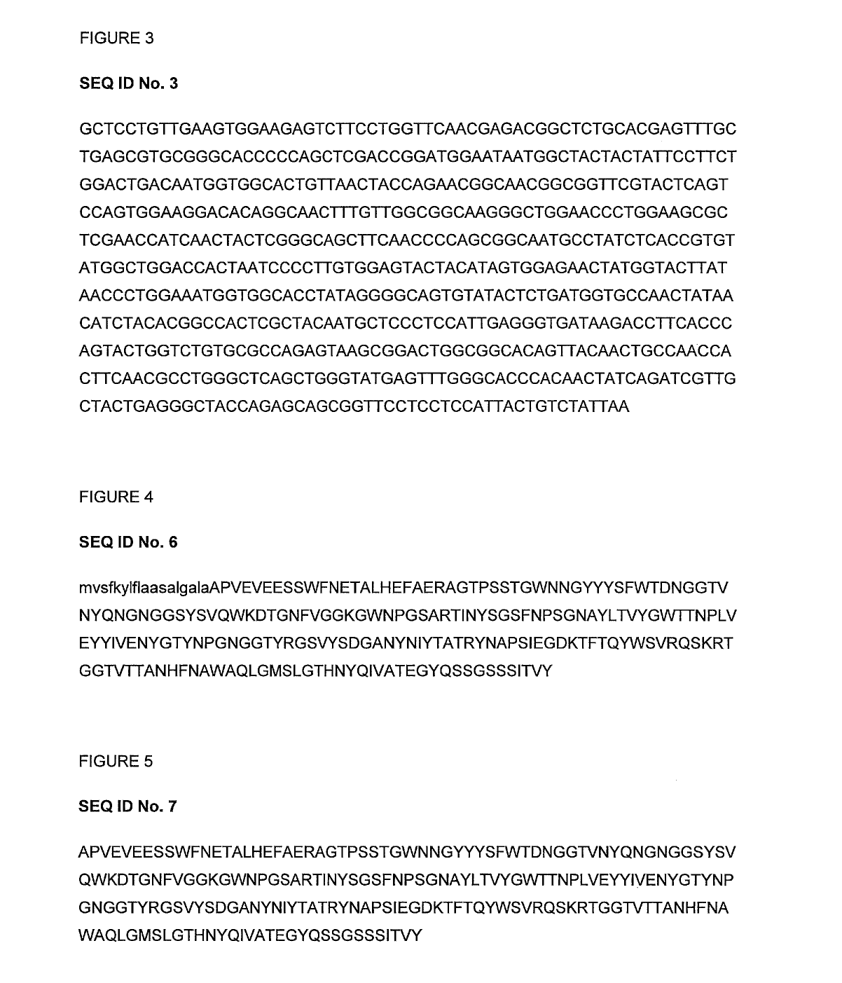

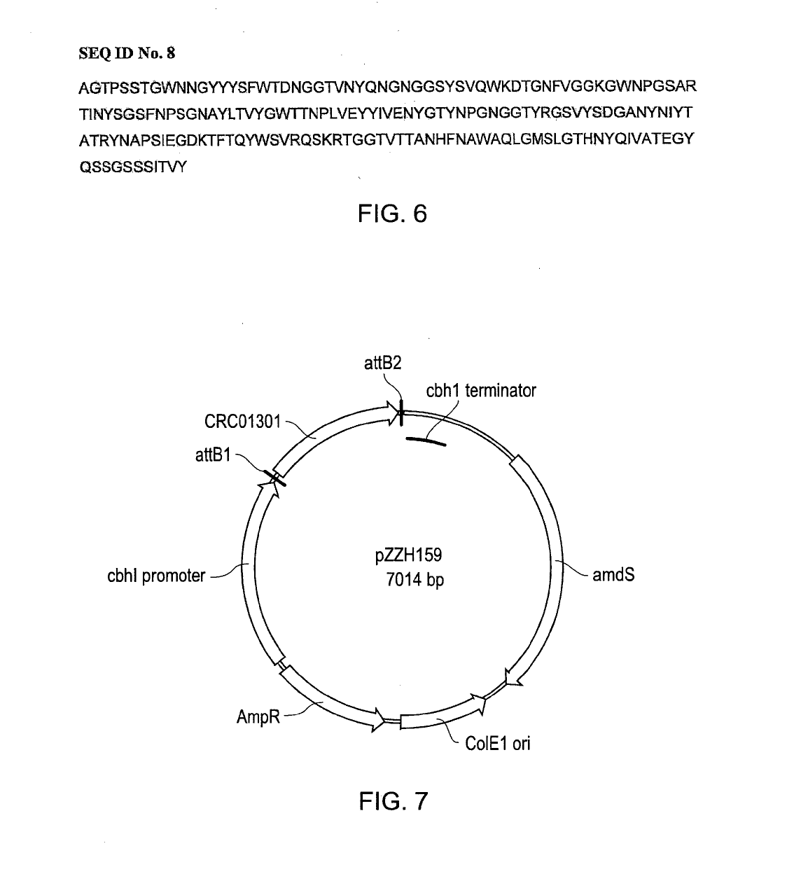

### SUMMARY OF THE INVENTION

A seminal finding of the present invention is that a specific xylanase from Aspergillus clavatus is surprisingly good at the solubilisation of saccharides (e.g. pentosans) in corn and/or corn by-products.

In particular the xylanase enzyme is unexpectedly good at breaking down (solubilising) insoluble arabinoxylans (AXinsol). Surprisingly the enzyme has been found to efficiently breakdown (solubilise) AXinsol from a wide range of substrates, including corn, wheat, DDGS, etc, in particular corn and corn based substrates, in particular both wheat (including wheat-based) products and corn (Including corn-based products). This contrasts with prior-known enzymes, which are often inferior at solubilising AXinsol in corn or corn-based substrates or which are not efficient in both wheat- and corn-based substrates.

In addition, the enzyme of the present invention is particularly good at not only breaking down (solubilising) AXinsol, but also breaking down (or degrading) the solubilized polymers efficiently.

Without wishing to be bound by theory, although some conventional xylanases breakdown AXinsol, they lead to an increase in soluble degradation products of high molecular weight, which may not be advantageous.

Furthermore or alternatively and again without wishing to be bound by theory, conventional xylanase enzymes may breakdown AXinsol, but because they do not degrade the solubilised products of high molecular weight fast enough the viscosity in the mixture is not ideal. In contrast, with the methods and uses of the present invention, the xylanases breakdown AXinsol whilst also quickly degrading the solubilised products of high molecular weight—thus providing improvements in digestibility, performance and feed efficiency in a subject compared with conventional enzymes.

The enzymes of the present invention and as described herein have been found to not only breakdown (solubilise) insoluble arabinoxylans (AXinsol) from a wide range of substrates, including corn, wheat, DDGS, etc, in particular corn and corn-based substrates, in particular both wheat (including wheat-based) products and corn (including corn-based products), but also efficiently breakdown the thus solubilised polymers.

Thus the present invention relates to enzymes capable of solubilising pentosans, in particular xylan-containing materials, such as arabinoxylans, in particular insoluble arabinoxylans. In particular the enzyme is particularly good at solubilising pentosans in particular xylan-containing materials, such as arabinoxylans, in particular insoluble arabinoxylans, in a broad spectrum of substrates, including corn based substrates.

In addition the heterogeneity/variance of hemicellulose from different sources is huge. While the underlying structure of most xylans is similar, i.e. α3-1,4 linked backbone of D-Xylose residues, in practice the variety is enormous due to differences in backbone size and in type and degree of substitutions from the backbone, all of which depend on the source of the xylan. The substitution pattern, in particular, can vary significantly from source to source, the substituents most commonly being α-4-0-methylglucuronic acid, arabinose, acetic acid, and various phenolics linked through substituent sugars. Substitution patterns in both xylans alter not only their physical properties, e.g. solubility, water binding capacity, viscosity, but also their susceptibility to attack by enzymes. For example, xylan hydrolysis products from corn are different from those produced from wheat in size, degree of substitution and in quantity. As a result of such heterogeneity in plant cell wall structure, a plethora of xylanolytic systems have evolved, each with their own characteristics. The present inventors have surprisingly developed a xylanase that is good at the solubilisation of saccharides (e.g. pentosans) in a broad spectrum of plant material and particularly in corn and/or corn by-products.

Based on these findings, the xylanases according to the present invention can be used to degrade a xylan-containing material, particularly arabinoxylans, particularly AXinsol. In addition or alternatively, the xylanases according to the present invention can be used to degrade soluble polymers (e.g. oligomers) that are produced from degradation of AXinsol or that are (naturally) present in grain-based materials. Surprisingly it has been found that the xylanases according the present invention can be used to both degrade a xylan-containing material, particularly arabinoxylans, particularly AXinsol, and to then degrade soluble polymers (e.g. oligomers) that are produced from degradation of AXinsol.

Based on this surprising finding the present invention provides a method of preparing feed and/or feed additive compositions which may result in increased solubilisation of arabinoxylan (such as pentosans) in corn or corn by-products.

For example, such feed and/or feed additive compositions may result in improved cost-efficiencies; improved performance of a subject; improved digestibility (e.g. nutrient digestibility) or improve feed efficiency in a subject; and/or improved yield.

### STATEMENTS OF THE INVENTION

According to a first aspect, the present invention relates to a method of preparing a corn based product said method comprising contacting a plant composition comprising (consisting of or consisting essentially of) corn and/or a corn by-product with a xylanase comprising a polypeptide as set forth in SEQ ID No. 6 or SEQ ID No. 7 or SEQ ID No. 8; or a variant, fragment, homologue or derivative thereof having at least 85% (suitably at least 90% or at least 95%) identity with SEQ ID No. 6 or SEQ ID No. 7 or SEQ ID No. 8;

or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% (suitably at least 85% or at least 90% or at least 95%) identity with SEQ ID No. 1, SEQ ID No. 2 or SEQ ID No. 3.

In one aspect, the corn based product may be a corn based feed product or a portion thereof.

In one aspect, the method may further comprise the addition of one or more additional plant materials, such as a high fibre plant material. For example, the method may further comprise the addition of one or more additional feed materials, such as a high fibre feed material.

Increasing prices of raw material traditionally used as an energy source (e.g. in animal feed) have resulted in inclusion of low-cost fibrous materials in the starting substrates for these industries, particularly the use of low-cost fibrous by-products in animal feed.

However, fibre addition may cause several disadvantageous effects. For example in animal feed fibre addition may cause anti-nutritional effects. The presence of un-degraded polymers present in the animal's intestine causes a highly viscous content and impeded diffusion with reduced nutrient absorption as a result. Also, the polymers possess a high water holding capacity hindering an effective re-absorption of water, and the water retention increases the volume of the gut content, which leads to a decrease intestinal transit time (Englyst & Kingman (1993) in Human Nutrition and Dietetics, 9th edition (Garrow J. S., James W. P. T., eds.) p. 53).

In feedstuffs, hemicellulose and cellulose also form physical barriers encapsulating nutrients like starch and protein and thereby retaining access to these nutrients for the animal.

Advantageously, the method of preparing a feedstuff or feed additive composition of the present invention can breakdown complex carbohydrates in low cost fibrous material such as corn DDGS solubilising saccharides such as pentosans. Accordingly, costs can be reduced whilst ameliorating or reducing detrimental effects associated with low cost fibrous materials. Furthermore, advantageously, the solubilisation of e.g. pentosans from such low cost (e.g. corn based) fibrous materials by the enzyme of the present invention can result in increased animal performance, feed efficacy and/or nutrient digestibility in a subject.

In one aspect, the feed material composition may be contacted with the xylanase by mixing the feed material composition with the xylanase, spraying the xylanase onto the feed material composition or dipping the food material composition into a preparation comprising the xylanase.

In one aspect, the corn by-product is corn gluten meal or corn gluten feed or corn Distillers Dried Grains (cDDG) or corn Distillers Dried Grain with Solubles (DDGS).

In one aspect, the feed product or feed material composition is a compound feed, a compound feed component, a premix of a compound feed, a fodder, a fodder component, or a premix of a fodder.

In one aspect, a method of preparing a feed product according to the present invention may comprise the step of forming the feed material composition into a meal, a pellet, a nut, a cake or a crumble

The present invention yet further provides a corn based product prepared in accordance with the method of the present invention.

In another aspect the present invention provides a corn based product comprising corn and/or a corn by-product and a xylanase comprising:
i) a polypeptide as set forth in SEQ ID No. 6 or SEQ ID No. 7 or SEQ ID No. 8; or a variant, fragment, homologue, fragments or derivative thereof having at least 85% identity with SEQ ID No. 6 or SEQ ID No. 7 or SEQ ID No. 8; or a xylanase encoded by:
   a) a nucleotide sequence shown herein as SEQ ID No. 1, SEQ ID No. 2 or SEQ ID No. 3, or
   b) a nucleotide sequence which can hybridize to the complement of SEQ ID No. 1, SEQ ID No. 2 or SEQ ID No. 3 under high stringency conditions, or
   c) a nucleotide sequence which has at least 80% identity with SEQ ID No. 1, SEQ ID No. 2 or SEQ ID No. 3.

The present invention further provides a method of preparing a feed additive composition, comprising admixing a xylanase comprising a polypeptide as set forth in SEQ ID No. 6 or SEQ ID No. 7 or SEQ ID No. 8; or a variant, fragment, homologue or derivative thereof having at least 85% identity with SEQ ID No. 6 or SEQ ID No. 7 or SEQ ID No. 8; or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% identity with SEQ ID No. 1, SEQ ID No. 2 or SEQ ID No. 3, with a feed acceptable carrier, diluent or excipient, and (optionally) packaging.

In another aspect there is provided a feed additive composition (or a packaged feed additive composition) comprising (or consisting essentially or of consisting of) a xylanase comprising a polypeptide as set forth in SEQ ID No. 6 or SEQ ID No. 7 or SEQ ID No. 8; or a variant, fragment, homologue or derivative thereof having at least 85% (suitably at least 90% or at least 95%) identity with SEQ ID No. 6 or SEQ ID No. 7 or SEQ ID No. 8; or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% (suitably at least 85% or at least 90% or at least 95%) identity with SEQ ID No. 1, SEQ ID No. 2 or SEQ ID No. 3 and a feed acceptable carrier, diluent or excipient.

In a yet further aspect the present invention provides a premix comprising a feed additive composition according to the present invention or a xylanase comprising a polypeptide as set forth in SEQ ID No. 6 or SEQ ID No. 7 or SEQ ID No. 8; or a variant, fragment, homologue or derivative thereof having at least 85% (suitably at least 90% or at least 95%) identity with SEQ ID No. 6 or SEQ ID No. 7 or SEQ ID No. 8; or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% (suitably at least 85% or at least 90% or at least 95%) identity with SEQ ID No. 1, SEQ ID No. 2 or SEQ ID No. 3; in combination with at least one mineral and/or at least one vitamin.

Suitably, a feed additive composition according to the present invention or a premix according to the present invention may be formulated as a dry powder or granules (preferably TPT granules).

In one aspect, the present invention provides a method of improving the performance of a subject or improving digestibility (e.g. nutrient digestibility) or improving feed efficiency in a subject comprising administering:
a. a corn based product prepared in accordance with the present invention; or
b. a feed additive composition or a premix according to the present invention; or
c. a xylanase comprising a polypeptide as set forth in SEQ ID No. 6 or SEQ ID No. 7 or SEQ ID No. 8; or a variant, fragment, homologue or derivative thereof having at least 85% identity with SEQ ID No. 6 or SEQ ID No. 7 or SEQ ID No. 8; or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% (suitably at least 85% or at least 90% or at least 95%) identity with SEQ ID No. 1, SEQ ID No. 2 or SEQ ID No. 3;
wherein in b. and c. the subject is optionally further administered a plant composition, e.g. a plant composition comprising corn or a corn by-product.

In one aspect, the present invention relates to the use of a corn based product in accordance with the present invention or a portion thereof, or a feed additive composition according to the present invention or a premix according to the present invention, or a xylanase comprising a polypeptide as set forth in SEQ ID No. 6 or SEQ ID No. 7 or SEQ ID No. 8; or a variant, fragment, homologue or derivative thereof having at least 85% (suitably at least 90% or at least 95%) identity with SEQ ID No. 6 or SEQ ID No. 7 or SEQ ID No. 8; or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% (suitably at least 85% or at least 90% or at least 95%) identity with SEQ ID No. 1, SEQ ID No. 2 or SEQ ID No. 3; to improve the performance of a subject or improve digestibility (e.g. nutrient digestibility) in a subject or improve feed efficiency in a subject, particularly in relation to corn-based feed products.

The present invention yet further provides a kit comprising a feed additive composition according to the present invention or a premix according to the present invention and instructions for administration with a corn-based feed product.

In a further aspect there is provided the use of a xylanase comprising a polypeptide as set forth in SEQ ID No. 6 or SEQ ID No. 7 or SEQ ID No. 8; or a variant, fragment, homologue or derivative thereof having at least 85% (suitably at least 90% or at least 95%) identity with SEQ ID No. 6 or SEQ ID No. 7 or SEQ ID No. 8; or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% (suitably at least 85% orleasteast 90% orleasteast 95%) identity with SEQ ID No. 1, SEQ ID No. 2 or SEQ ID No. 3, in the production of a fermented beverage, such as a beer.

In a yet further aspect, the present invention provides a method of producing a fermented beverage (e.g. beer) comprising the step of contacting a mash and/or a wort with a xylanase comprising a polypeptide as set forth in SEQ ID No. 6 or SEQ ID No. 7 or SEQ ID No. 8; or a variant, fragment, homologue or derivative thereof having at least 85% (suitably at least 90% or at least 95%) identity with SEQ ID No. 6 or SEQ ID No. 7 or SEQ ID No. 8; or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% (suitably at least 85% or at least 90% or at least 95%) identity with SEQ ID No. 1, SEQ ID No. 2 or SEQ ID No. 3.

A further aspect of the present invention provides a method of producing a fermented beverage (e.g. beer) comprising the steps of: (a) preparing a mash, (b) filtering the mash to obtain a wort, and (c) fermenting the wort to obtain a fermented beverage, such as a beer, wherein a xylanase comprising a polypeptide as set forth in SEQ ID No. 6 or SEQ ID No. 7 or SEQ ID No. 8; or a variant, fragment, homologue or derivative thereof having at least 85% (suitably at least 90% or at least 95%) identity with SEQ ID No. 6 or SEQ ID No. 7 or SEQ ID No. 8; or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% (suitably at least 85% or at least 90% or at least 95%) identity with SEQ ID No. 1, SEQ ID No. 2 or SEQ ID No. 3, is added to: (i) the mash of step (a) and/or (ii) the wort of step (b) and/or (iii) the wort of step (c).

The present invention yet further provides a fermented beverage, such as a beer, produced by a method of present invention.

For the avoidance of doubt, SEQ ID No. 8 is the mature form of SEQ ID No. 6 or SEQ ID No. 7. SEQ ID No. 8 is the form of the protein which arises following posttranslational processing. SEQ ID No. 7 is also an active form of the enzyme and may also be referred to herein as the mature form of the enzyme. Therefore all of these sequences relate to the same enzyme.

This enzyme is encoded by the nucleotide sequences shown herein as SEQ ID Nos. 1, 2 and 3.

### DETAILED DISCLOSURE OF THE PREFERRED EMBODIMENTS OF THE INVENTION

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure which can be had by reference to the specification as a whole. Accordingly, the terms defined immediately below are more fully defined by reference to the specification as a whole.

Amino acids are referred to herein using the name of the amino acid, the three letter abbreviation or the single letter abbreviation.

The term “protein”, as used herein, includes proteins, polypeptides, and peptides.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to understand that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “an enzyme” includes a plurality of such candidate agents and reference to “the feed” includes reference to one or more feeds and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

In one aspect, the xylanase enzyme for use in the method, uses and compositions of the present invention may be obtainable from (or obtained from) a fungus, namely Aspergillus clavatus.

In one aspect the present invention provides a xylanase obtainable from (or obtained from) Aspergillus clavatus for use in a corn based feed or a feed additive composition.

The xylanase enzyme of the present invention may be referred to herein as AclXyn5.

The term “Hemicellulose”—as used herein means the polysaccharide components of plant cell walls other than cellulose. The term “hemicellulose” as used herein may mean polysaccharides in plant cell walls which are extractable by dilute alkaline solutions. Hemicelluloses comprise almost one-third of the carbohydrates in woody plant tissue. The chemical structure of hemicelluloses consists of long chains of a variety of pentoses, hexoses, and their corresponding uronic acids. Hemicelluloses may be found in fruit, plant stems, and grain hulls. The polysaccharides yielding pentoses on complete hydrolysis are called pentosans. Xylan is an example of a pentosan consisting of D-xylose units with 1β→4 linkages.

The term “pentosan” as used herein is any of a group of carbohydrates which yield pentoses on complete hydrolysis.

The term “arabinoxylans” (AX) as used herein means a polysaccharide found in the bran of grains such as wheat, maize (corn), rye, and barley consisting of a xylan backbone (1,4-linked xylose units) with L-arabinofuranose (L-arabinose in its 5-atom ring form) attached randomly by 1α→2 and/or 1α→3 linkages to the xylose units throughout the chain. Arabinoxylan is a hemicellulose found in both the primary and secondary cell walls of plants.

Since xylose and arabinose (the constituents of arabinoxylans) are both pentoses, arabinoxylans are usually classified as pentosans.

The term “consisting essentially of” as used herein means that unspecified components may be present if the characteristics of the claimed composition are thereby not materially affected.

The term “consisting of” means that the proportions of the specific ingredients must total 100%.

The term “comprising” used herein may be amended in some embodiments to refer to consisting essentially of or consisting of (both having a more limited meaning that “comprising”).

### Advantages

The use of xylanase taught herein has many advantages compared with known xylanases.

The xylanases as taught herein are unexpectedly good at solubilising pentosans, particularly in corn based products.

The xylanases as taught herein are unexpectedly good at solubilising AXinsol, particularly in corn based products.

In particular the xylanase of the present invention is unexpectedly good at degrading pentosans, in particular in breaking down xylan-containing materials, such as arabinoxylans (e.g. AXinsol) in corn based substrates. Compared with a benchmark xylanase which is a commercially produced and marketed xylanase, the xylanase taught herein is capable of much more efficient degradation and pentosan release from corn-based substrates compared with the marketed xylanases. This was completely unexpected. This gives the xylanase of the present invention applicability in a broad range of applications.

Surprisingly it has been found that the xylanase of the present invention is particularly good at degrading xylan-containing materials, such as arabinoxylans, e.g. AXinsol, in a broad spectrum of substrates, corn, wheat, DDGS, etc, in particular corn and corn based substrates, in particular both wheat (including wheat-based) products and corn (including corn-based products). Compared with the benchmark xylanases which are all commercially produced and marketed xylanases, the novel xylanase taught herein was capable of much more efficient degradation and pentosan release from more plant based materials (in particular corn-based substrates) compared with the marketed xylanases. This was completely unexpected. This contrasts with prior-known enzymes, which are often inferior at solubilising AXinsol in comrn or corn-based substrates or which are not as efficient in both wheat- and corn-based substrates.

In addition, the enzyme of the present invention is particularly good at not only breaking down (solubilising) AXinsol, but also breaking down (or degrading) the solubilized polymers efficiently.

The enzyme of the present invention is particularly effective at enhancing the performance of a subject or improving the digestibility of a raw material in a corn based feedstuff and/or for improving feed efficiency in a subject.

### Plant Composition

The term “plant composition” as used herein means a plant composition comprising (consisting of or consisting essentially of) corn and/or a corn by-product.

In one embodiment the plant composition, corn and/or corn by-product is a feedstuff or feed component.

### Corn-Based Product

The term “corn based product” as used herein means a plant composition which comprises (or consists essentially of or consists of) corn (maize) seed or grain or a by-product of corn grain.

The corn-based product may be a corn-based feedstuff or a corn-based starting material for malting or brewing.

Preferably the corn based product or the plant composition comprises corn or a by-product of corn as the major constituent. For example the corn based product or the plant composition may comprise at least 35% corn or a by-product of corn, such as at least 50% corn or a by-product of corn, such as at least 70% or a by-product of corn, such as at least 90% corn or a by-product of corn, for example 100% corn or a by-product of corn.

In some embodiments the corn based product or the plant composition may comprise corn or a by-product of corn as a minor constituent; in which case the feedstuff may be supplemented with corn or a by-product of corn. By way of example only the corn based product or the plant composition may comprise for example wheat supplemented with corn or a by-product of corn.

When corn or the by-product of corn is a minor constituent of the corn based product or the plant composition, the corn or by-product of corn is at least 20%, preferably at least 30%, preferably at least 40%, preferably at least 50% of the feedstuff.

For the avoidance of doubt the term “corn” as used herein is synonymous with maize, e.g. Zea mays.

In one embodiment the by-product of corn may be corn gluten meal, or corn Distillers Dried Grain Solubles (cDDGS).

### Feed or Feedstuff

In one aspect, the corn based product may be a corn based feed product or a portion thereof. When the corn based product is used as a portion of a corn based feed product, the corn based product may be considered a feed additive composition. Therefore in some embodiments the term corn based product as used herein may mean a feed product or feed additive composition.

The feed additive composition of the present invention may be used as—or in the preparation of—a feed.

The term “feed” is used synonymously herein with “feedstuff”. The term “feedstuff” as used herein means food suitable for animal consumption, such as for cattle (e.g. cows), pigs, sheep (e.g. lambs), goats, Poultry, such as chickens or laying hens, turkeys, ostriches, pheasants, deer, elk, reindeer, buffalo, bison, antelope, camels, kangaroos; horses, fish; cats, dogs, guinea pigs, rodents e.g. rats, mice, gerbils and chinchillas.

The feed may be in the form of a solution or as a solid or as a semi-solid—depending on the use and/or the mode of application and/or the mode of administration.

When used as—or in the preparation of—a feed—such as functional feed—the enzyme or composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

In one aspect, the feed material composition comprises (or consists essentially of or consists of) corn (maize) or a corn by-product. In one aspect, the feed material composition is a feed or feed additive composition.

In one aspect, the feed additive composition of the present invention is admixed with a feed component to form a feedstuff.

The term “feed component” as used herein means all or part of the feedstuff. Part of the feedstuff may mean one constituent of the feedstuff or more than one constituent of the feedstuff, e.g. 2 or 3 or 4. In one embodiment the term “feed component” encompasses a premix or premix constituents.

Preferably the feed may be a fodder, or a premix thereof, a compound feed, or a premix thereof. In one embodiment the feed additive composition according to the present invention may be admixed with a compound feed, a compound feed component or to a premix of a compound feed or to a fodder, a fodder component, or a premix of a fodder.

The term fodder as used herein means any food which is provided to an animal (rather than the animal having to forage for it themselves). Fodder encompasses plants that have been cut.

The term fodder includes silage, compressed and pelleted feeds, oils and mixed rations, and also sprouted grains and legumes.

Fodder may be obtained from one or more of the plants selected from: corn (maize), alfalfa (Lucerne), barley, birdsfoot trefoil, brassicas, Chau moellier, kale, rapeseed (canola), rutabaga (swede), turnip, clover, alsike clover, red clover, subterranean clover, white clover, fescue, brome, millet, oats, sorghum, soybeans, trees (pollard tree shoots for tree-hay), wheat, and legumes.

The term “cornpound feed” means a commercial feed in the form of a meal, a pellet, nuts, cake or a crumble. Compound feeds may be blended from various raw materials and additives. These blends are formulated according to the specific requirements of the target animal.

Compound feeds can be complete feeds that provide all the daily required nutrients, concentrates that provide a part of the ration (protein, energy) or supplements that only provide additional micronutrients, such as minerals and vitamins.

The main ingredients used in compound feed are the feed grains, which include corn, wheat canola meal, rapeseed meal, lupin, soybeans, sorghum, oats, rye and barley.

Suitably a premix as referred to herein may be a composition composed of microingredients such as vitamins, minerals, chemical preservatives, antibiotics, fermentation products, and other essential ingredients. Premixes are usually compositions suitable for blending into commercial rations.

Any feedstuff of the present invention may in addition to comprising corn or a corn by-product further comprise one or more feed materials selected from the group comprising a) cereals, such as small grains (e.g., wheat, barley, rye, oats and combinations thereof) and/or large grains such as sorghum; b) by products from cereals, such as gluten meal, Distillers Dried Grain Solubles (DDGS)), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; e) minerals and vitamins.

In one embodiment the feed component may be corn, DDGS (e.g. cDDGS), corn gluten meal, or a combination thereof.

In one embodiment the feedstuff comprises or consists of corn, DDGS (such as cDDGS), corn gluten meal, or a combination thereof.

In one embodiment a feed component may be corn, DDGS (such as cDDGS) or a combination thereof.

A feedstuff of the present invention may contain at least 30%, at least 40%, at least 50% or at least 60% by weight corn and/or corn by-product.

In addition or in the alternative, a feedstuff of the present invention may comprise at least one high fibre feed material and/or at least one by-product of the at least one high fibre feed material to provide a high fibre feedstuff. Examples of high fibre feed materials include: wheat, barley, rye, oats, by products from cereals, such as corn gluten meal, Distillers Dried Grain Solubles (DDGS), wheat bran, wheat middlings, wheat shorts, rice bran, rice hulls, oat hulls, palm kernel, and citrus pulp. Some protein sources may also be regarded as high fibre: protein obtained from sources such as canola, sunflower, lupin, fava beans and cotton.

In one embodiment the feedstuff of the present invention comprises at least one high fibre material and/or at least one by-product of the at least one high fibre feed material selected from the group consisting of Distillers Dried Grain Solubles (DDGS)—particularly cDDGS, wheat bran, and wheat for example.

In the present invention the feed may be one or more of the following: a compound feed and premix, including pellets, nuts or (cattle) cake; a crop or crop residue: corn, soybeans, sorghum, oats, barley, copra, chaff, sugar beet waste; fish meal; meat and bone meal; molasses; oil cake and press cake; oligosaccharides; conserved forage plants: silage; seaweed; seeds and grains, either whole or prepared by crushing, milling etc.; sprouted grains and legumes; yeast extract.

The term feed in the present invention also encompasses in some embodiments pet food. A pet food is plant or animal material intended for consumption by pets, such as dog food or cat food. Pet food, such as dog and cat food, may be either in a dry form, such as kibble for dogs, or wet canned form. Cat food may contain the amino acid taurine.

The term feed in the present invention also encompasses in some embodiments fish food. A fish food normally contains macro nutrients, trace elements and vitamins necessary to keep captive fish in good health. Fish food may be in the form of a flake, pellet or tablet Pelleted forms, some of which sink rapidly, are often used for larger fish or bottom feeding species.

Some fish foods also contain additives, such as beta carotene or sex hormones, to artificially enhance the color of ornamental fish.

The term feed in the present invention also encompasses in some embodiment bird food. Bird food includes food that is used both in birdfeeders and to feed pet birds. Typically bird food comprises of a variety of seeds, but may also encompass suet (beef or mutton fat).

In one aspect, the feed is for livestock such as pigs, sheep, cows and poultry.

In one aspect, the feed is poultry feed.

As used herein the term “contacted” refers to the indirect or direct application of the enzyme (or composition comprising the enzyme) of the present invention to the product (e.g. the feed). Examples of the application methods which may be used, include, but are not limited to, treating the product in a material comprising the feed additive composition, direct application by mixing the feed additive composition with the product, spraying the feed additive composition onto the product surface or dipping the product into a preparation of the feed additive composition.

In one embodiment the feed additive composition of the present invention is preferably admixed with the product (e.g. feedstuff). Alternatively, the feed additive composition may be included in the emulsion or raw ingredients of a feedstuff.

For some applications, it is important that the composition is made available on or to the surface of a product to be affected/treated. This allows the composition to impart one or more of the following favourable characteristics: performance benefits.

The enzyme (or composition comprising the enzyme) of the present invention may be applied to intersperse, coat and/or impregnate a product (e.g. feedstuff or raw ingredients of a feedstuff) with a controlled amount of said enzyme.

Preferably, the enzyme for use in the present invention (or composition comprising the enzyme of the present invention) is formulated to be thermally stable to heat treatment up to about 70° C.; up to about 85° C.; or up to about 95° C. The heat treatment may be performed for up to about 1 minute; up to about 5 minutes; up to about 10 minutes; up to about 30 minutes; up to about 60 minutes. The term thermally stable means that at least about 75% of the enzyme that was present/active in the additive before heating to the specified temperature is still present/active after it cools to room temperature. Preferably, at least about 80% of the enzyme that is present and active in the additive before heating to the specified temperature is still present and active after it cools to room temperature.

In a particularly preferred embodiment the enzyme for use in the present invention (or composition comprising the enzyme of the present invention) is homogenized to produce a powder.

In an alternative preferred embodiment, the enzyme (or composition comprising the enzyme) of the present invention is formulated to granules as described in WO2007/044968 (referred to as TPT granules) or WO1997/016076 or WO1992/012645 incorporated herein by reference.

In another preferred embodiment when the feed additive composition is formulated into granules the granules comprise a hydrated barrier salt coated over the protein core. The advantage of such salt coating is improved thermo-tolerance, improved storage stability and protection against other feed additives otherwise having adverse effect on the enzyme.

Preferably, the salt used for the salt coating has a water activity greater than 0.25 or constant humidity greater than 60% at 20° C.

Preferably, the salt coating comprises a Na₂SO₄.

The method of preparing an enzyme for use in the present invention (or composition comprising the enzyme of the present invention) may also comprise the further step of pelleting the powder. The powder may be mixed with other components known in the art. The powder, or mixture comprising the powder, may be forced through a die and the resulting strands are cut into suitable pellets of variable length.

Optionally, the pelleting step may include a steam treatment, or conditioning stage, prior to formation of the pellets. The mixture comprising the powder may be placed in a conditioner, e.g. a mixer with steam injection. The mixture is heated in the conditioner up to a specified temperature, such as from 60-100° C., typical temperatures would be 70° C., 80° C., 85° C., 90° C. or 95° C. The residence time can be variable from seconds to minutes and even hours. Such as 5 seconds, 10 seconds, 15 seconds, 30 seconds, 1 minutes 2 minutes, 5 minutes, 10 minutes, 15 minutes, 30 minutes and 1 hour.

It will be understood that the enzyme for use in the present invention (or composition comprising the enzyme of the present invention) is suitable for addition to any appropriate feed material (e.g. comprising corn or a corn by-product).

It will be understood by the skilled person that different animals require different feedstuffs, and even the same animal may require different feedstuffs, depending upon the purpose for which the animal is reared.

Optionally, the feedstuff may also contain additional minerals such as, for example, calcium and/or additional vitamins.

Preferably, the feedstuff is a corn soybean meal mix.

In one embodiment, preferably the feed is not pet food.

In another aspect there is provided a method for producing a feedstuff. Feedstuff is typically produced in feed mills in which raw materials are first ground to a suitable particle size and then mixed with appropriate additives. The feedstuff may then be produced as a mash or pellets; the later typically involves a method by which the temperature is raised to a target level and then the feed is passed through a die to produce pellets of a particular size. The pellets are allowed to cool. Subsequently liquid additives such as fat and enzyme may be added. Production of feedstuff may also involve an additional step that includes extrusion or expansion prior to pelleting—in particular by suitable techniques that may include at least the use of steam.

The feedstuff may be a feedstuff for a monogastric animal, such as poultry (for example, broiler, layer, broiler breeders, turkey, duck, geese, water fowl), and swine (all age categories), a ruminant such as cattle (e.g. cows or bulls (including calves)), horses, sheep, a pet (for example dogs, cats) or fish (for example agastric fish, gastric fish, freshwater fish such as salmon, cod, trout and carp, e.g. koi carp, marine fish such as sea bass, and crustaceans such as shrimps, mussels and scallops). Preferably the feedstuff is for poultry.

### Feed Additive Composition

The feed additive composition of the present invention and/or the feedstuff comprising same may be used in any suitable form.

The feed additive composition of the present invention may be used in the form of solid or liquid preparations or alternatives thereof. Examples of solid preparations include powders, pastes, boluses, capsules, pellets, tablets, dusts, and granules which may be wettable, spray-dried or freeze-dried. Examples of liquid preparations include, but are not limited to, aqueous, organic or aqueous-organic solutions, suspensions and emulsions.

In some applications, the feed additive compositions of the present invention may be mixed with feed or administered in the drinking water.

In one aspect the present invention relates to a method of preparing a feed additive composition, comprising admixing a xylanase as taught herein with a feed acceptable carrier, diluent or excipient, and (optionally) packaging.

### Premix

The feedstuff and/or feed additive composition may be combined with at least one mineral and/or at least one vitamin. The compositions thus derived may be referred to herein as a premix.

### Corn Based Feedstuff

In a preferred embodiment the feedstuff may be a corn based feedstuff. The term “corn based feedstuff” as used herein means a feedstuff which comprises or consists of corn (maize) or a by-product of corn.

Preferably the corn based feedstuff comprises corn or a by-product of corn as the major constituent. For example the corn based feedstuff may comprise at least 35% corn or a by-product of corn, such as at least 40% corn or a by-product of corn, such as at least 50% corn or a by-product of corn, such as at least 60% corn or a by-product of corn, such as at least 70% corn or a by-product of corn, such as at least 80% or a by-product of corn, such as at least 90% corn or a by-product of corn, for example 100% corn or a by-product of corn.

In some embodiments the corn based feedstuff may comprise corn or a by-product of corn as a minor constituent; in which case the feedstuff may be supplemented with corn or a by-product of corn. By way of example only the feedstuff may comprise for example wheat supplemented with corn or a by-product of corn.

When corn or the by-product of corn is a minor constituent of the feedstuff, the corn or by-product of corn is at least 5%, preferably at least 10%, preferably at least 20%, preferably at least 30% of the feedstuff.

For the avoidance of doubt the term “corn” as used herein is synonymous with maize, e.g. Zea mays.

In one embodiment the by-product of corn may be corn Distillers Dried Grain Solubles (cDDGS) or corn wet-cake or corn Distillers Dried Grain (DDG) or corn gluten meal or combinations thereof.

In one embodiment preferably feedstuff or corn by-product of the present invention comprises a by-product of corn, such as corn Distillers Dried Grain Solubles (cDDGS) or corn wet-cake or corn Distillers Dried Grain (DDG) or corn gluten meal or combinations thereof.

Corn by-Product

A corn by-product may be any product derived from corn or the processing of corn.

Examples of corn by-products are any arabinoxylan-containing material which is a by-product of corn, such as corn Distillers Dried Grain Solubles (cDDGS) or corn wet-cake or corn Distillers Dried Grain (DDG) or corn gluten meal or combinations thereof.

### Wet-Cake, Distillers Dried Grains (DDG) and Distillers Dried Grain Solubles (DDGS)

Wet-cake, Distillers Dried Grains and Distillers Dried Grains with Solubles are products obtained after the removal of ethyl alcohol by distillation from yeast fermentation of a grain or a grain mixture by methods employed in the grain distilling industry.

Stillage coming from the distillation (e.g. comprising water, remainings of the grain, yeast cells etc.) is separated into a “solid” part and a liquid part.

The solid part is called “wet-cake” and can be used as animal feed as such.

The liquid part is (partially) evaporated into a syrup (solubles).

When the wet-cake is dried it is Distillers Dried Grains (DDG).

When the wet-cake is dried together with the syrup (solubles) it is Distillers Dried Grans with Solubles (DDGS).

Wet-cake may be used in dairy operations and beef cattle feedlots.

The dried DDGS may be used in livestock, e.g. dairy, beef and swine) feeds and poultry feeds.

Corn DDGS is a very good protein source for dairy cows.

### Corn Gluten Meal

In one aspect, the by-product of corn may be corn gluten meal (CGM).

CGM is a powdery by-product of the corn milling industry. CGM has utility in, for example, animal feed. It can be used as an inexpensive protein source for feed such as pet food, livestock feed and poultry feed. It is an especially good source of the amino acid cysteine, —but must be balanced with other proteins for lysine.

### Malting and Brewing

The enzyme (or composition comprising the enzyme) of the present invention may be used in the production of a fermented beverage, such as beer and/or in malting and brewing.

When the xylanase is used in the production of a fermented beverage, such as beer, and/or in malting or brewing the xylanase may be contacted with a mash and/or a wort (said mash and/or said wort may be produced from barley or wheat).

Efficient hydrolysis of arabinoxylans (AXsol) and beta-glucan is important because such compounds can be involved in production problems such as wort viscosity (Ducroo, P. & Frelon, P. G., Proceedings of the European Brewery Convention Congress, Zurich, 1989, 445; Viëtor, R. J. & Voragen, A. G. J., Journal of the Institute of Brewing, 1993, 99, 243) and filterability and haze formation (Coote, N. & Kirsop, B. H. 1976, Journal of the Institute of Brewing, 1976, 82, 34; Izawa, M., Kano, Y. & Kanimura, M. 1991. Proceedings Aviemore Conference on Malting, brewing and Distillling, 1990, 427).

The present invention provides a method of hydrolysing arabinoxylans (e.g. AXinsol and AXsol) during malting and brewing wherein grain-material, a mash, a wort, an adjunct, a malt, a portion thereof, or a combination thereof, are admixed with the enzyme of the present invention.

In one aspect the grain-material, the mash, the wort, the adjunct, the malt, the portion thereof, or the combination thereof, are obtained from barley or wheat.

In one aspect of the present invention may relate to a food composition that is a beverage, including, but not limited to, a fermented beverage such as beer and wine, comprising a xylanase comprising a polypeptide as set forth in SEQ ID No. 6 or SEQ ID No. 7 or SEQ ID No. 8; or a variant, fragment, homologue or derivative thereof having at least 85% identity with SEQ ID No. 6 or SEQ ID No. 7 or SEQ ID No. 8; or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% identity with SEQ ID No. 1, SEQ ID No. 2 or SEQ ID No. 3.

In the context of the present invention, the term “fermented beverage” is meant to comprise any beverage produced by a method comprising a fermentation process, such as a microbial fermentation, such as a bacterial and/or yeast fermentation.

In an aspect of the invention the fermented beverage is beer. The term “beer” is meant to comprise any fermented wort produced by fermentation/brewing of a starch-containing plant material. Often, beer is produced from malt or adjunct, or any combination of malt and adjunct as the starch-containing plant material. As used herein the term “malt” is understood as any malted cereal grain, such as malted barley or wheat.

As used herein the term “adjunct” refers to any starch and/or sugar containing plant material which is not malt, such as barley or wheat malt. As examples of adjuncts, mention can be made of materials such as common corn grits, refined corn grits, brewer's milled yeast, rice, sorghum, refined corn starch, barley, barley starch, dehusked barley, wheat, wheat starch, torrified cereal, cereal flakes, rye, oats, corn (maize), potato, tapioca, cassava and syrups, such as corn syrup, sugar cane syrup, inverted sugar syrup, barley and/or wheat syrups, and the like may be used as a source of starch.

As used herein, the term “mash” refers to an aqueous slurry of any starch and/or sugar containing plant material such as grist, e. g. comprising crushed barley malt, crushed barley, and/or other adjunct or a combination hereof, mixed with water later to be separated into wort and spent grains.

As used herein, the term “wort” refers to the unfermented liquor run-off following extracting the grist during mashing.

In another aspect the invention relates to a method of preparing a fermented beverage such as beer comprising mixing the xylanase of the present invention with malt or adjunct.

Examples of beers comprise: full malted beer, beer brewed under the “Reinheitsgebot”, ale, IPA, lager, bitter, Happoshu (second beer), third beer, dry beer, near beer, light beer, low alcohol beer, low calorie beer, porter, bock beer, stout, malt liquor, non-alcoholic beer, non-alcoholic malt liquor and the like, but also alternative cereal and malt beverages such as fruit flavoured malt beverages, e. g. citrus flavoured, such as lemon-, orange-, lime-, or berry-flavoured malt beverages, liquor flavoured malt beverages, e.g., vodka-, rum-, or tequila-flavoured malt liquor, or coffee flavoured malt beverages, such as caffeine-flavoured malt liquor, and the like.

### Xylan-Containing Material

The xylanase for use in the methods and uses of the present invention (or composition comprising the xylanase for use in the methods and uses of the present invention) may be used to degrade any xylan-containing material.

Hence the plant composition, corn and/or corn by-product comprises xylan-containing material.

In one embodiment the plant composition, corn and/or corn by-product comprises insoluble arabinoxylan (AXinsol).

### Breakdown or Degradation

The enzyme (or composition comprising the enzyme) of the present invention or as disclosed herein may be used to breakdown (degrade) AXinsol or AXsol or degradation products of AXinsol in a plant composition, corn based product, corn, corn by-product or feedstuff. The term “breakdown” or “degrade” in synonymous with hydrolyses.

### Solubilisation/Degradation

The present invention relates to a method of preparing a corn based product, such as a feed or feed additive composition comprising corn.

Suitably, the present invention may relate to the degradation of a xylan-containing material (preferably an arabinoxylan-containing material, preferably an insoluble arabinoxylan (AXinsol)-containing material) to produce soluble pentosans (which can be polymeric, oligomeric or monomeric) in a plant composition, corn based products, corn, corn by-products or feedstuffs.

This method may be described herein as pentosan solubilisation or arabinoxylan solubilisation or AXinsol solubilisation or degradation of AXinsol.

In one embodiment, the present invention relates to a method of degrading (or breaking down) insoluble arabinoxylan (AXinsol). This can also be referred to as solubilisation of Insoluble arabinoxylan and/or solubilisation of pentosans.

In a further embodiment of the present invention the method relates to degrading (e.g. breaking down) polymers derived from the degradation of insoluble arabinoxylans.

Suitably, this method may involve degrading a corn based product or a plant composition comprising corn to produce saccharides such as C5 and C6 sugars (preferably, pentosans such as xylose).

Suitably, this method may involve degrading a xylan-containing material present in corn (preferably an arabinoxylan-containing material) to produce saccharides such as C5 and C6 sugars (preferably, pentosans such as xylose).

The term “solubilistation” as used herein refers to the degradation of a xylan-containing material present in corn (preferably an arabinoxylan-containing material) to produce saccharides such as C5 and C6 sugars (preferably. pentosans such as xylose).

The solubilisation of pentosans can be measured by the following assay:

### Quantification of C5 Sugars (Pentosans)

The total amount of pentoses brought into solution was measured using the method of Rouau and Surget (1994, A rapid semi-automated method of the determination of total and water-extractable pentosan in wheat flours. Carbohydrate Polymers, 24, 123-32) with a continuous flow injection apparatus (FIG. 7). The supematants were treated with acid to hydrolyse polysaccharides to monosugars. Phloroglucinol (1,3,5-trihydroxybenzen) was added for reaction with monopentoses and monohexoses, which forms a coloured complex. By measuring the difference in absorbance at 550 nm compared to 510 nm, the amount of pentoses in the solution was calculated using a standard curve. Unlike the pentose-phloroglucinol complex, the absorbance of the hexose-phloroglucinol complex is constant at these wavelengths. Glucose was added to the phloroglucinol solution to create a constant glucose signal and further ensure no interference from hexose sugars.

An increase in solubilisation as used herein may mean an increase in the solubilisation (e.g. release) of pentosans, e.g. measure in accordance with an assay taught herein.

In one embodiment, an increase in solubilisation by use of the enzyme(s) of the present invention means an increase in pentosan release of between 5× and 15× (suitably between 6× and 10×) compared with the pentosan release observed in a control without enzyme addition.

In one embodiment, an increase in solubilisation by use of the enzyme(s) of the present invention means an increase in pentosan release of at least 5× (preferably at least 6×, more preferably at least 10× or suitably at least 15×) that of the pentosan release observed in a control without enzyme addition.

### Arabinoxylan (AX)

The term “arabinoxylans” (AX) as used herein means a polysaccharide consisting of a xylan backbone (1,4-linked xylose units) with L-arabinofuranose (L-arabinose in its 5-atom ring form) attached randomly by 1α→2 and/or 1α→3 linkages to the xylose units throughout the chain. Arabinoxylan is a hemicellulose found in both the primary and secondary cell walls of plants. Arabinoxylan can be found in the bran of grasses and grains such as wheat, maize (corn), rye, and barley.

Arabinoxylan (AX) is found in close association with the plant cell wall, where it acts as a glue linking various building blocks of the plant cell wall and tissue, give it both structural strength and rigidity.

The term “pentosan” as used herein a polysaccharide composes of mainly pentoses.

Since xylose and arabinose (the constituents of arabinoxylans) are both pentoses, arabinoxylans are usually classified as pentosans.

AX is the principal Non Starch Polysaccharide (NSP)-fraction in several of the most important feed raw material, including wheat and corn.

Its abundance, location within vegetable material and molecular structure cause AX to have a severe, negative impact on feed digestibility, effectively reducing the nutritional value of the raw materials in which it is present. This makes AX an important anti-nutritional factor, reducing animal production efficiency.

The term “Hemicellulose”—as used herein means the polysaccharide components of plant cell walls other than cellulose. The term “hemicellulose” as used herein may mean polysaccharides in plant cell walls which are extractable by dilute alkaline solutions. Hemicelluloses comprise almost one-third of the carbohydrates in woody plant tissue. The chemical structure of hemicelluloses consists of long chains of a variety of pentoses, hexoses, and their corresponding uronic acids. Hemicelluloses may be found in fruit, plant stems, and grain hulls. Xylan is an example of a pentosan consisting of D-xylose units with 1β→4 linkages.

### Water Insoluble Arabinoxylan (AXinsol)

Water-insoluble arabinoxylan (AXinsol) also known as water-unextractable arabinoxylan (WU-AX) constitutes a significant proportion of the dry matter of plant material.

In corn AXinsol can account for 3.5-6% (e.g. 5.1%) of the dry matter. In corn DDGS AXinsol can account for 10-20% (e.g. 12.6%) of the dry matter.

AXinsol causes nutrient entrapment in feed. Large quantities of well digestible nutrients such as starch and proteins remain either enclosed in clusters of cell wall material or bound to side chains of the AX. These entrapped nutrients will not be available for digestion and subsequent absorption in the small intestine.

### Water-Soluble Arabinoxylan (AXsol)

In feed water-soluble arabinoxylan (AXsol) can have an anti-nutritional effect particularly in monogastrics as they can cause a considerable increase of the viscosity of the intestinal content, caused by the extraordinary water-binding capacity of AXsol. The increased viscosity can affect feed digestion and nutrient use as it can prevent proper mixing of feed with digestive enzymes and bile salts and/or it slows down nutrient availability and absorption and/or it stimulates fermentation in the hindgut.

In corn AXsol can account for 0.1-0.4% (e.g. 0.1%) of the dry matter. In corn DDGS AXinsol can account for 0.3-2.5% (e.g. 0.4%) of the dry matter.

In addition, however, to the amount of AXsol present in plant material, when a xylanase solubilises AXinsol in the plant material this can release pentosans and/or oligomers which contribute to AXsol content of the plant material.

One advantage of the xylanases disclosed herein is that they have the ability to both solubilise AXinsol as well as to rapidly and efficiently breakdown the solubilised oligomers and/or pentosans thus the enzymes are able to solubilise AXinsol.

A breakdown of AXsol can release nutrients.

### Feed Ingredient

The feed additive composition of the present invention may be used as a feed ingredient.

As used herein the term “feed ingredient” includes a formulation which is or can be added to functional feeds or feedstuffs as a nutritional supplement and/or fibre supplement. The term feed ingredient as used here also refers to formulations which can be used at low levels in a wide variety of products that require gelling, texturising, stabilising, suspending, film-forming and structuring, retention of juiciness and improved mouthfeel, without adding viscosity.

The feed ingredient may be in the form of a solution or as a solid—depending on the use and/or the mode of application and/or the mode of administration.

### Xylanases

In one aspect the xylanase for use in the methods, uses, compositions and/or corn based products (e.g. feed) of present invention is a xylanase comprising or consisting of:
i) a polypeptide as set forth in SEQ ID No. 6 or SEQ ID No. 7 or SEQ ID No. 8; or a variant, fragment, homologue or derivative thereof having at least 85% (suitably at least 90% or at least 95%) identity with SEQ ID No. 6 or SEQ ID No. 7 or SEQ ID No. 8; or
is a xylanase encoded by:
   a) a nucleotide sequence shown herein as SEQ ID No. 1, SEQ ID No. 2 or SEQ ID No. 3, or
   b) a nucleotide sequence which can hybridize to the complement of SEQ ID No. 1, SEQ ID No. 2 or SEQ ID No. 3 under high stringency conditions, or
   c) a nucleotide sequence which has at least 80% (suitably at least 85% or at least 90% or at least 95%) identity with SEQ ID No. 1, SEQ ID No. 2 or SEQ ID No. 3.

Suitably, the xylanase may comprising or consisting of a polypeptide having at least 90%, at least 95%, at least 96%, at least 97%, at least 98% or at least 99% identity to SEQ ID No. 6 or SEQ ID No. 7 or SEQ ID No. 8.

Suitably, the xylanase may comprise or consist of a polypeptide encoded by a nucleotide sequence having at least 85%, at least 90%, at least 95%, at least 96%, at least 97%, at least 98% or at least 99% identity to SEQ ID No. 1 or SEQ ID No. 2 or SEQ ID No. 3.

### Dosages

Preferably, the xylanase is present in the plant composition or corn based product (e.g. feedstuff, feed material composition or feed additive composition) in the range of about 500 XU/kg to about 16,000 XU/kg composition/product (e.g. feed), more preferably about 750 XU/kg composition/product to about 8000 XU/kg composition/product (e.g. feed), preferably about 1500 XU/kg feed to about 3000 XU/kg xylan-containing material (e.g. feed), preferably about 2000 XU/kg feed to about 2500 XU/kg xylan-containing material (e.g. feed), and even more preferably about 1000 XU/kg composition/product (e.g. feed) to about 4000 XU/kg composition/product (e.g. feed).

In one embodiment the xylanase is present in the plant composition or corn based product (e.g. feedstuff) at more than about 500 XU/kg composition/product (e.g. feed), suitably more than about 600 XU/kg composition/product (e.g. feed), suitably more than about 700 XU/kg composition/product (e.g. feed), suitably more than about 800 XU/kg composition/product (e.g. feed), suitably more than about 900 XU/kg composition/product (e.g. feed), suitably more than about 1000 XU/kg composition/product (e.g. feed), suitably more than about 2000 XU/kg, suitably more than about 2500 XU/kg, suitably more than about 3000 XU/kg, suitably more than about 3500 XU/kg, suitably more than about 4000 XU/kg xylan-containing material (e.g. feed).

In one embodiment the xylanase is present in the plant composition or corn based product (e.g. feedstuff) at less than about 16,000 XU/kg composition/product (e.g. feed), suitably less than about 8000 XU/kg composition/product (e.g. feed), suitably less than about 7000 XU/kg composition/product (e.g. feed), suitably less than about 6000 XU/kg composition/product (e.g. feed), suitably less than about 5000 XU/kg composition/product (e.g. feed), suitably less than about 4000 XU/kg composition/product (e.g. feed).

Preferably, the xylanase may be present in a feed additive composition in range of about 100 XU/g to about 320,000 XU/g composition, more preferably about 300 XU/g composition to about 160,000 XU/g composition, and even more preferably about 500 XU/g composition to about 50,000 XU/g composition, and even more preferably about 500 XU/g composition to about 40,000 XU/g composition.

In one embodiment the xylanase is present in the feed additive composition at more than about 100 XU/g composition, suitably more than about 200 XU/g composition, suitably more than about 300 XU/g composition, suitably more than about 400 XU/g composition, suitably more than about 500 XU/g composition.

In one embodiment the xylanase is present in the feed additive composition at less than about 320,000 XU/g composition, suitably less than about 160,000 XU/g composition, suitably less than about 50,000 XU/g composition, suitably less than about 40,000 XU/g composition, suitably less than about 30000 XU/g composition.

The xylanase activity can be expressed in xylanase units (XU) measured at pH 5.0 with AZCL-arabinoxylan (azurine-crosslinked wheat arabinoxylan, Xylazyme tablets, Megazyme) as substrate. Hydrolysis by endo-(1-4)-ß-D-xylanase (xylanase) produces water soluble dyed fragments, and the rate of release of these (increase in absorbance at 590 nm) can be related directly to enzyme activity. The xylanase units (XU) are determined relatively to an enzyme standard (Danisco xylanase, available from Danisco Animal Nutrition) at standard reaction conditions, which are 40° C., 5 min reaction time in Mclivaine buffer, pH 5.0.

The xylanase activity of the standard enzyme is determined as amount of released reducing sugar end groups from an oat-spelt-xylan substrate per min at pH 5.3 and 50° C. The reducing sugar end groups react with 3, 5-Dinitrosalicylic acid and formation of the reaction product can be measured as increase in absorbance at 540 nm. The enzyme activity is quantified relative to a xylose standard curve (reducing sugar equivalents). One xylanase unit (XU) is the amount of standard enzyme that releases 0.5 μmol of reducing sugar equivalents per min at pH 5.3 and 50° C.

In one embodiment suitably the enzyme is classified using the E.C. classification above, and the E.C. classification designates an enzyme having that activity when tested in the assay taught herein for determining 1 XU.

The dose of the enzyme in the feed product or feed additive composition according to the present invention may be designed for one-time dosing or may be designed for use (e.g. feeding) on a daily basis.

The optimum amount of the enzyme and/or composition comprising the enzyme to be used in the present invention will depend on the product to be treated and/or the method of contacting the product with the composition and/or the intended use for the same.

The amount of enzyme used in the compositions should be a sufficient amount to be effective.

The amount of enzyme used in the compositions should be a sufficient amount to be effective and to remain sufficiently effective in for example improving the performance of an animal fed feed products containing said composition. This length of time for effectiveness should extend up to at least the time of utilisation of the product (e.g. feed additive composition or feed containing same).

### Formulation

In one embodiment the enzyme may be formulated as a liquid, a dry powder or a granule.

The dry powder or granules may be prepared by means known to those skilled in the art, such as, in top-spray fluid bed coater, in a buttom spray Wurster or by drum granulation (e.g. High sheer granulation), extrusion, pan coating or in a microingredients mixer.

For some embodiments the enzyme may be coated, for example encapsulated.

In one embodiment the coating protects the enzyme from heat and may be considered a thermoprotectant.

In one embodiment the feed additive composition is formulated to a dry powder or granules as described in WO2007/044968 (referred to as TPT granules) or WO1997/016076 or WO1992/012645 (each of which is incorporated herein by reference).

In one embodiment the feed additive composition may be formulated to a granule for feed compositions comprising: a core; an active agent; and at least one coating, the active agent of the granule retaining at least 50% activity, at least 60% activity, at least 70% activity, at least 80% activity after conditions selected from one or more of a) a feed pelleting process, b) a steam-heated feed pretreatment process, c) storage, d) storage as an ingredient in an unpelleted mixture, and e) storage as an ingredient in a feed base mix or a feed premix comprising at least one compound selected from trace minerals, organic acids, reducing sugars, vitamins, choline chloride, and compounds which result in an acidic or a basic feed base mix or feed premix.

With regard to the granule at least one coating may comprise a moisture hydrating material that constitutes at least 55% w/w of the granule; and/or at least one coating may comprise two coatings. The two coatings may be a moisture hydrating coating and a moisture barrier coating. In some embodiments, the moisture hydrating coating may be between 25% and 60% w/w of the granule and the moisture barrier coating may be between 2% and 15% w/w of the granule. The moisture hydrating coating may be selected from inorganic salts, sucrose, starch, and maltodextrin and the moisture barrier coating may be selected from polymers, gums, whey and starch.

The granule may be produced using a feed pelleting process and the feed pretreatment process may be conducted between 70° C. and 95° C. for up to several minutes, such as between 85° C. and 95° C.

In one embodiment the feed additive composition may be formulated to a granule for animal feed comprising: a core; an active agent, the active agent of the granule retaining at least 80% activity after storage and after a steam-heated pelleting process where the granule is an ingredient; a moisture barrier coating; and a moisture hydrating coating that is at least 25% w/w of the granule, the granule having a water activity of less than 0.5 prior to the steam-heated pelleting process.

The granule may have a moisture barrier coating selected from polymers and gums and the moisture hydrating material may be an inorganic salt. The moisture hydrating coating may be between 25% and 45% w/w of the granule and the moisture barrier coating may be between 2% and 10% w/w of the granule.

The granule may be produced using a steam-heated pelleting process which may be conducted between 85° C. and 95° C. for up to several minutes.

In some embodiments the enzyme or feed additive composition may be diluted using a diluent, such as starch powder, lime stone or the like.

In one embodiment, the enzyme or feed additive composition comprising the enzyme is in a liquid formulation suitable for consumption preferably such liquid consumption contains one or more of the following: a buffer, salt, sorbitol and/or glycerol.

In another embodiment the enzyme or feed additive composition comprising the enzyme may be formulated by applying, e.g. spraying, the enzyme(s) onto a carrier substrate, such as ground corn for example.

In one embodiment the enzyme or feed additive composition comprising the enzyme according to the present invention may be formulated as a premix. By way of example only the premix may comprise one or more feed components, such as one or more minerals and/or one or more vitamins.

In one embodiment the enzyme for use in the present invention is formulated with at least one physiologically acceptable carrier selected from at least one of maltodextrin, limestone (calcium carbonate), cyclodextrin, wheat or a wheat component, sucrose, starch, Na₂SO₄, Talc, PVA, sorbitol, benzoate, sorbiate, glycerol, sucrose, propylene glycol, 1,3-propane diol, glucose, parabens, sodium chloride, citrate, acetate, phosphate, calcium, metabisulfite, formate and mixtures thereof.

### Packaging

In one embodiment the corn based product (e.g. feed product, feed additive composition) or a portion thereof is packaged.

In one preferred embodiment the feed additive composition and/or premix and/or feed or feedstuff is packaged in a bag, such as a paper bag.

In an alternative embodiment the feed additive composition and/or premix and/or feed or feedstuff may be sealed in a container. Any suitable container may be used.

### Forms

The enzyme fur use in the present invention or composition comprising the enzyme (e.g. the feed additive composition) of the present invention and other components and/or or feedstuff comprising same may be used in any suitable form.

Suitable forms include (or preferably are) in the form of solid or liquid preparations or alternatives thereof. Examples of solid preparations include powders, pastes, boluses, capsules, pellets, tablets, pills, capsules, ovules, solutions or suspensions, dusts, and granules which may be wettable, spray-dried or freeze-dried. Examples of liquid preparations include, but are not limited to, aqueous, organic or aqueous-organic solutions, suspensions and emulsions.

The composition comprising the enzyme may contain flavouring or colouring agents, for immediate-, delayed-, modified-, sustained-, pulsed- or controlled-release applications.

By way of example, if the composition of the present invention is used in a solid, e.g. pelleted form, it may also contain one or more of: excipients such as microcrystalline cellulose, lactose, sodium citrate, calcium carbonate, dibasic calcium phosphate and glycine; disintegrants such as starch (preferably cornm, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

Preferred excipients for the forms include lactose, starch, a cellulose, milk sugar or high molecular weight polyethylene glycols.

For aqueous suspensions and/or elixirs, the composition of the present invention may be combined with various sweetening or flavouring agents, colouring matter or dyes, with emulsifying and/or suspending agents and with diluents such as water, propylene glycol and glycerin, and combinations thereof.

### Subject

The term “subject”, as used herein, means an animal that is to be or has been administered with a feed additive composition according to the present invention or a feedstuff comprising said feed additive composition according to the present invention.

The term “subject”, as used herein, means an animal.

In one embodiment, the subject is a mammal, bird, fish or crustacean including for example livestock or a domesticated animal (e.g. a pet).

In one embodiment the “subject” is livestock.

The term “livestock”, as used herein refers to any farmed animal. Preferably, livestock is one or more of ruminants such as cows or bulls (including calves), mono-gastric animals such as poultry (including broilers, chickens and turkeys), pigs (including piglets), birds, aquatic animals such as fish, agastric fish, gastric fish, freshwater fish such as salmon, cod, trout and carp, e.g. koi carp, marine fish such as sea bass, and crustaceans such as shrimps, mussels and scallops), horses (including race horses), sheep (including lambs).

In another embodiment the “subject” is a domesticated animal or pet or an animal maintained in a zoological environment.

The term “domesticated animal or pet or animal maintained in a zoological environment” as used herein refers to any relevant animal including canines (e.g. dogs), felines (e.g. cats), rodents (e.g. guinea pigs, rats, mice), birds, fish (including freshwater fish and marine fish), and horses.

### Performance

As used herein, “animal performance” may be determined by the feed efficiency and/or weight gain of the animal and/or by the feed conversion ratio and/or by the digestibility of a nutrient in a feed (e.g. amino acid digestibility) and/or digestible energy or metabolizable energy in a feed and/or by nitrogen retention and/or by animals ability to avoid the negative effects of necrotic enteritis and/or by the immune response of the subject.

Preferably “animal performance” is determined by feed efficiency and/or weight gain of the animal and/or by the feed conversion ratio.

By “improved animal performance” it is meant that there is increased feed efficiency, and/or increased weight gain and/or reduced feed conversion ratio and/or improved digestibility of nutrients or energy in a feed and/or by improved nitrogen retention in the subject resulting from the use of feed additive composition of the present invention in feed in comparison to feed which does not comprise said feed additive composition.

Preferably, by “improved animal performance” it is meant that there is increased feed efficiency and/or increased weight gain and/or reduced feed conversion ratio.

As used herein, the term “feed efficiency” refers to the amount of weight gain per unit of feed when the animal is fed ad-libitum or a specified amount of food during a period of time.

By “increased feed efficiency” it is meant that the use of a feed additive composition according the present invention in feed results in an increased weight gain per unit of feed intake compared with an animal fed without said feed additive composition being present.

### Feed Conversion Ratio (FCR)

As used herein, the term “feed conversion ratio” refers to the amount of feed fed to an animal to increase the weight of the animal by a specified amount.

An improved feed conversion ratio means a lower feed conversion ratio.

By “lower feed conversion ratio” or “improved feed conversion ratio” it is meant that the use of a feed additive composition in feed results in a lower amount of feed being required to be fed to an animal to increase the weight of the animal by a specified amount compared to the amount of feed required to increase the weight of the animal by the same amount when the feed does not comprise said feed additive composition.

### Nutrient Digestibility

Nutrient digestibility as used herein means the fraction of a nutrient that disappears from the gastro-intestinal tract or a specified segment of the gastro-intestinal tract, e.g. the small intestine. Nutrient digestibility may be measured as the difference between what is administered to the subject and what comes out in the faeces of the subject, or between what is administered to the subject and what remains in the digesta on a specified segment of the gastro intestinal tract, e.g. the ileum.

Nutrient digestibility as used herein may be measured by the difference between the intake of a nutrient and the excreted nutrient by means of the total collection of excreta during a period of time; or with the use of an inert marker that is not absorbed by the animal, and allows the researcher calculating the amount of nutrient that disappeared in the entire gastro-intestinal tract or a segment of the gastro-intestinal tract. Such an inert marker may be titanium dioxide, chromic oxide or acid insoluble ash. Digestibility may be expressed as a percentage of the nutrient in the feed, or as mass units of digestible nutrient per mass units of nutrient in the feed.

Nutrient digestibility as used herein encompasses starch digestibility, fat digestibility, protein digestibility, and amino acid digestibility.

Energy digestibility as used herein means the gross energy of the feed consumed minus the gross energy of the faeces or the gross energy of the feed consumed minus the gross energy of the remaining digesta on a specified segment of the gastro-intestinal tract of the animal, e.g. the ileum. Metabolizable energy as used herein refers to apparent metabolizable energy and means the gross energy of the feed consumed minus the gross energy contained in the faeces, urine, and gaseous products of digestion. Energy digestibility and metabolizable energy may be measured as the difference between the intake of gross energy and the gross energy excreted in the faeces or the digesta present in specified segment of the gastro-intestinal tract using the same methods to measure the digestibility of nutrients, with appropriate corrections for nitrogen excretion to calculate metabolizable energy of feed.

Reduced Waste Output from Animal Production

The xylanase of the present invention can be used to reduce waste output from animal production. This has significant advantages.

Combination with Other Components

The enzyme for use in the present invention may be used in combination with other components.

In one embodiment the enzyme for use in the present invention may be used in combination with a probiotic or a direct fed microbial (DFM), e.g. a direct fed bacteria.

The combination of the present invention comprises the enzyme of the present invention (or a composition comprising the enzyme, e.g. a feed additive composition) and another component which is suitable for human or animal consumption and is capable of providing a medical or physiological benefit to the consumer.

In one embodiment the “another component” may be one or more further enzymes (e.g. further feed).

Suitable additional enzymes for use in the present invention may be one or more of the enzymes selected from the group consisting of: endoglucanases (E.C. 3.2.1.4); celliobiohydrolases (E.C. 3.2.1.91), Pglucosidases (E.C. 3.2.1.21), cellulases (E.C. 3.2.1.74), lichenases (E.C. 3.1.1.73), lipases (E.C. 3.1.1.3), lipid acytransferases (generally classified as E.C. 2.3.1.x), phospholipases (E.C. 3.1.1.4, E.C. 3.1.1.32 or E.C. 3.1.1.5), phytases (e.g. 6-phytase (E.C. 3.1.3.26) or a 3-phytase (E.C. 3.1.3.8), alpha-amylases (E.C. 3.2.1.1), other xylanases (E.C. 3.2.1.8, E.C. 3.2.1.32, E.C. 3.2.1.37, E.C. 3.1.1.72, E.C. 3.1.1.73), glucoamylases (E.C. 3.2.1.3), proteases (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)) and/or mannanases (e.g. a 3-mannanase (E.C. 3.2.1.78)).

In one embodiment (particularly for feed applications) the other component may be one or more of the enzymes selected from the group consisting of an amylase (including α-amylases (E.C. 3.2.1.1), G4-forming amylases (E.C. 3.2.1.60), β-amylases (E.C. 3.2.1.2) and γ-amylases (E.C. 3.2.1.3); and/or a protease (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)).

In one embodiment (particularly for feed applications) the other component may be a combination of an amylase (e.g. α-amylases (E.C. 3.2.1.1)) and a protease (e.g. subtilisin (E.C. 3.4.21.62)).

In one embodiment (particularly for feed applications) the other component may be a β-glucanase, e.g. an endo-1,3(4)-β-glucanases (E.C. 3.2.1.6).

In one embodiment (particularly for feed applications) the other component may be a mannanases (e.g. a β-mannanase (E.C. 3.2.1.78)).

In one embodiment (particularly for feed applications) the other component may be a lipase lipase (E.C. 3.1.1.3), a lipid acyltransferase (generally classified as E.C. 2.3.1.x), or a phospholipase (E.C. 3.1.1.4, E.C. 3.1.1.32 or E.C. 3.1.1.5), suitably a lipase (E.C. 3.1.1.3).

In one embodiment (particularly for feed applications) the other component may be a protease (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)).

In one embodiment the additional component may be a stabiliser or an emulsifier or a binder or carrier or an excipient or a diluent or a disintegrant.

The term “stabiliser” as used here is defined as an Ingredient or combination of Ingredients that keeps a product (e.g. a feed product) from changing over time.

The term “emulsifier” as used herein refers to an ingredient (e.g. a feed ingredient) that prevents the separation of emulsions. Emulsions are two immiscible substances, one present in droplet form, contained within the other. Emulsions can consist of oil-in-water, where the droplet or dispersed phase is oil and the continuous phase is water; or water-in-oil, where the water becomes the dispersed phase and the continuous phase is oil. Foams, which are gas-in-liquid, and suspensions, which are solid-in-liquid, can also be stabilised through the use of emulsifiers.

As used herein the term “binder” refers to an ingredient (e.g. a feed ingredient) that binds the product together through a physical or chemical reaction. During “gelation” for instance, water is absorbed, providing a binding effect. However, binders can absorb other liquids, such as oils, holding them within the product. In the context of the present invention binders would typically be used in solid or low-moisture products for instance baking products: pastries, doughnuts, bread and others. Examples of granulation binders include one or more of: polyvinylpyrrolidone, hydroxypropylmethylcellulose (HPMC), hydroxypropylcellulose (HPC), sucrose, maltose, gelatin and acacia.

“Carriers” mean materials suitable for administration of the enzyme and include any such material known in the art such as, for example, any liquid, gel, solvent, liquid diluent, solubilizer, or the like, which is non-toxic and which does not interact with any components of the composition in a deleterious manner.

The present invention provides a method for preparing a composition (e.g. a feed additive composition) comprising admixing an enzyme of the present invention (and preferably corn or a corn by-product) with at least one physiologically acceptable carrier selected from at least one of maltodextrin, limestone (calcium carbonate), cyclodextrin, wheat or a wheat component, sucrose, starch, Na₂SO₄, Talc, PVA, sorbitol, benzoate, sorbiate, glycerol, sucrose, propylene glycol, 1,3-propane diol, glucose, parabens, sodium chloride, citrate, acetate, phosphate, calcium, metabisulfite, formate and mixtures thereof.

Examples of “excipients” include one or more of microcrystalline cellulose and other celluloses, lactose, sodium citrate, calcium carbonate, dibasic calcium phosphate, glycine, starch, milk sugar and high molecular weight polyethylene glycols.

Examples of “disintegrants” include one or more of: starch (preferably corn, potato or tapioca starch), sodium starch glycollate, croscarmellose sodium and certain complex silicates.

Examples of “diluents” include one or more of water, ethanol, propylene glycol and glycerin, and combinations thereof.

The other components may be used simultaneously (e.g. when they are in admixture together or even when they are delivered by different routes) or sequentially (e.g. they may be delivered by different routes) to the xylanase of the present invention.

Preferably, when the feed additive composition of the present invention is admixed with another component(s), the DFM remains viable.

In one embodiment preferably the feed additive composition according to the present invention does not comprise chromium or organic chromium

In one embodiment preferably the feed additive according to the present invention does not contain glucanase.

In one embodiment preferably the feed additive according to the present invention does not contain sorbic acid.

The publications discussed herein are provided solely for their disclosure prior to the filing date of the present application. Nothing herein is to be construed as an admission that such publications constitute prior art to the claims appended hereto.

### Isolated

In one aspect, preferably the enzyme for use in the present invention is in an isolated form. The term “isolated” means that the enzyme is at least substantially free from at least one other component with which the enzyme is naturally associated in nature and as found in nature. The enzyme for use in the present invention may be provided in a form that is substantially free of one or more contaminants with which the substance might otherwise be associated. Thus, for example it may be substantially free of one or more potentially contaminating polypeptides and/or nucleic acid molecules.

### Purified

In one aspect, preferably the enzyme for use in the present invention is in a purified form. The term “purified” means that the given component is present at a high level. The component is desirably the predominant component present in a composition. Preferably, it is present at a level of at least about 90%, or at least about 95% or at least about 98%, said level being determined on a dry weight/dry weight basis with respect to the total composition under consideration.

### Nucleotide Sequence

The scope of the present invention encompasses the use of nucleotide sequences encoding proteins having the specific properties as defined herein.

The term “nucleotide sequence” as used herein refers to an oligonucleotide sequence or polynucleotide sequence, and variant, fragment, homologues, fragments and derivatives thereof (such as portions thereof). The nucleotide sequence may be of genomic or synthetic or recombinant origin, which may be double-stranded or single-stranded whether representing the sense or anti-sense strand.

The term “nucleotide sequence” in relation to the present invention includes genomic DNA, cDNA, synthetic DNA, and RNA. Preferably it means DNA, more preferably cDNA sequence coding for the present invention.

In a preferred embodiment, the nucleotide sequence when relating to and when encompassed by the per se scope of the present invention does not include the native nucleotide sequence according to the present invention when in its natural environment and when it is linked to its naturally associated sequence(s) that is/are also in its/their natural environment. For ease of reference, we shall call this preferred embodiment the “non-native nucleotide sequence”. In this regard, the term “native nucleotide sequence” means an entire nucleotide sequence that is in its native environment and when operatively linked to an entire promoter with which it is naturally associated, which promoter is also in its native environment. However, the amino acid sequence encompassed by scope the present invention can be isolated and/or purified post expression of a nucleotide sequence in its native organism. Preferably, however, the amino acid sequence encompassed by scope of the present invention may be expressed by a nucleotide sequence in its native organism but wherein the nucleotide sequence is not under the control of the promoter with which it is naturally associated within that organism.

Typically, the nucleotide sequence encompassed by the scope of the present invention is prepared using recombinant DNA techniques (i.e. recombinant DNA). However, in an alternative embodiment of the invention, the nucleotide sequence could be synthesised, in whole or in part, using chemical methods well known in the art (see Caruthers M H et al., (1980) Nuc Acids Res Symp Setr 215-23 and Horn T et al., (1980) Nuc Acids Res Symp Ser 225-232).

### Preparation of the Nucleotide Sequence

A nucleotide sequence encoding either a protein which has the specific properties as defined herein or a protein which is suitable for modification may be identified and/or isolated and/or purified from any cell or organism producing said protein. Various methods are well known within the art for the identification and/or isolation and/or purification of nucleotide sequences. By way of example, PCR amplification techniques to prepare more of a sequence may be used once a suitable sequence has been identified and/or isolated and/or purified.

By way of further example, a genomic DNA and/or cDNA library may be constructed using chromosomal DNA or messenger RNA from the organism producing the enzyme. If the amino acid sequence of the enzyme is known, labelled oligonucleotide probes may be synthesised and used to identify enzyme-encoding clones from the genomic library prepared from the organism. Alternatively, a labelled oligonucleotide probe containing sequences homologous to another known enzyme gene could be used to identify enzyme-encoding clones. In the latter case, hybridisation and washing conditions of lower stringency are used.

Alternatively, enzyme-encoding clones could be identified by inserting fragments of genomic DNA into an expression vector, such as a plasmid, transforming enzyme-negative bacteria with the resulting genomic DNA library, and then plating the transformed bacteria onto agar plates containing a substrate for enzyme (i.e. maltose), thereby allowing clones expressing the enzyme to be identified.

In a yet further alternative, the nucleotide sequence encoding the enzyme may be prepared synthetically by established standard methods, e.g. the phosphoroamidite method described by Beucage S. L. et al., (1981) Tetrahedron Letters 22, p 1859-1869, or the method described by Matthes et al., (1984) EMBO J. 3, p 801-805. In the phosphoroamidite method, oligonucleotides are synthesised, e.g. In an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al., (Science (1988) 239, pp 487-491).

### Amino Acid Sequences

The scope of the present invention also encompasses the use of amino acid sequences of enzymes having the specific properties as defined herein.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”̂.

The amino acid sequence may be prepared/isolated from a suitable source, or it may be made synthetically or it may be prepared by use of recombinant DNA techniques.

The protein encompassed in the present invention may be used in conjunction with other proteins, particularly enzymes. Thus the present invention also covers a combination of proteins wherein the combination comprises the protein/enzymê of the present invention and another protein/enzymê, which may be another protein/enzymê according to the present invention. This aspect is discussed in a later section.

Preferably the amino acid sequence when relating to and when encompassed by the per se scope of the present invention is not a native enzyme. In this regard, the term “native enzyme” means an entire enzyme that is in its native environment and when it has been expressed by its native nucleotide sequence.

### Sequence Identity or Sequence Homology

The present invention also encompasses the use of sequences having a degree of sequence identity or sequence homology with amino acid sequence(s) of a polypeptide having the specific properties defined herein or of any nucleotide sequence encoding such a polypeptide (hereinafter referred to as a “homologous sequence(s)”). Here, the term “homologue” means an entity having a certain homology with the subject amino acid sequences and the subject nucleotide sequences. Here, the term “homology” can be equated with “identity”.

The homologous amino acid sequence and/or nucleotide sequence should provide and/or encode a polypeptide which retains the functional activity and/or enhances the activity of the enzyme.

In the present context, a homologous sequence is taken to include an amino acid or a nucleotide sequence which may be at least 75, 85 or 90% identical, preferably at least 95 or 98% identical to the subject sequence. Typically, the homologues will comprise the same active sites etc. as the subject amino acid sequence for instance. Although homology can also be considered in terms of similarity (i.e. amino acid residues having similar chemical properties/functions), in the context of the present invention it is preferred to express homology in terms of sequence identity.

In one embodiment, a homologous sequence is taken to include an amino acid sequence or nucleotide sequence which has one or several additions, deletions and/or substitutions compared with the subject sequence.

In the present context, “the subject sequence” relates to the nucleotide sequence or polypeptide/amino acid sequence according to the invention.

Preferably, the % sequence identity with regard to a polypeptide sequence is determined using SEQ ID No. 7 as the subject sequence in a sequence alignment. In one embodiment, the subject sequence is selected from the group consisting of SEQ ID No. 6, 7 or 8. In a preferred embodiment the subject sequence is selected from the mature sequences SEQ ID No. 7.

Preferably, the % sequence identity with regard to a nucleotide sequence is determined using SEQ ID No. 3 as the subject sequence in the sequence alignment. In one embodiment, the subject sequence for nucleotide sequences may be selected from the group consisting of SEQ ID No. 1, 2 or 3. In a preferred embodiment the subject sequence is sequence SEQ ID No. 3.

A “parent nucleic acid” or “parent amino acid” means a nucleic acid sequence or amino acid sequence, encoding or coding for the parent polypeptide, respectively.

In one embodiment the present invention relates to a protein whose amino acid sequence is represented herein or a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

Suitably, the degree of identity with regard to an amino acid sequence is determined over at least 20 contiguous amino acids, preferably over at least 30 contiguous amino acids, preferably over at least 40 contiguous amino acids, preferably over at least 50 contiguous amino acids, preferably over at least 60 contiguous amino acids, preferably over at least 100 contiguous amino acids, preferably over at least 200 contiguous amino acids.

In one embodiment the present invention relates to a nucleic acid sequence (or gene) encoding a protein whose amino acid sequence is represented herein or encoding a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

In the present context, a homologous sequence is taken to include a nucleotide sequence or foreign sequence which may be at least 75, 85 or 90% identical, preferably at least 95 or 98% identical to a nucleotide sequence encoding a polypeptide of the present invention (the subject sequence). Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology or % identity between two or more sequences. % homology or % identity may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology or % identity when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum % homology or % identity therefore firstly requires the production of an optimal alignment, taking into consideration gap penalties. A suitable computer program for carrying out such an alignment is the Vector NTI (Invitrogen Corp.). Examples of software that can perform sequence comparisons include, but are not limited to, the BLAST package (see Ausubel et al 1999 Short Protocols in Molecular Biology, 4th Ed—Chapter 18), BLAST 2 (see FEMS Microbiol Lett 1999 174(2): 247-50; FEMS Microbiol Lett 1999 177(1): 187-8 and tatiana@ncbi.nlm.nih.gov), FASTA (Altschul et al 1990 J. Mol. Biol. 403-410) and AlignX for example. At least BLAST, BLAST 2 and FASTA are available for offline and online searching (see Ausubel et al 1999, pages 7-58 to 7-60), such as for example in the GenomeQuest search tool (www.genomequestcom).

Although the final % homology or % identity can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI package.

Alternatively, percentage homologies may be calculated using the multiple alignment feature in Vector NTI (Invitrogen Corp.), based on an algorithm, analogous to CLUSTAL (Higgins D G & Sharp P M (1988), Gene 73(1), 237-244).

Once the software has produced an optimal alignment, it is possible to calculate % homology, preferably % sequence identity. The software typically does this as part of the sequence comparison and generates a numerical result.

Should Gap Penalties be used when determining sequence identity, then preferably the following parameters are used for pairwise alignment:

| FOR BLAST | |
|---|---|
| GAP OPEN | 9 |
| GAP EXTENSION | 2 |

| FOR CLUSTAL | DNA | PROTEIN |
|---|---|---|
| Weight Matrix | IUB | Gonnet 250 |
| GAP OPENING | 15 | 10 |
| GAP EXTEND | 6.66 | 0.1 |

In one embodiment, CLUSTAL may be used with the gap penalty and gap extension set as defined above.

Suitably, the degree of identity with regard to a nucleotide sequence or protein sequence is determined over at least 20 contiguous nucleotides/amino acids, preferably over at least 30 contiguous nucleotides/amino acids, preferably over at least 40 contiguous nucleotides/amino acids, preferably over at least 50 contiguous nucleotides/amino acids, preferably over at least 60 contiguous nucleotides/amino acids, preferably over at least 100 contiguous nucleotides/amino acids.

Suitably, the degree of identity with regard to a nucleotide sequence is determined over at least 100 contiguous nucleotides, preferably over at least 200 contiguous nucleotides, preferably over at least 300 contiguous nucleotides, preferably over at least 400 contiguous nucleotides, preferably over at least 500 contiguous nucleotides, preferably over at least 600 contiguous nucleotides.

Suitably, the degree of identity with regard to a nucleotide sequence may be determined over the whole sequence taught herein.

Suitably, the degree of identity with regard to a nucleotide sequence may be determined over the whole sequence taught herein as the mature sequence, e.g. SEQ ID No. 3.

Suitably, the degree of identity with regard to a protein (amino acid) sequence is determined over at least 100 contiguous amino acids, preferably over at least 150 contiguous amino acids.

Suitably, the degree of identity with regard to an amino acid or protein sequence may be determined over the whole sequence taught herein.

Suitably, the degree of identity with regard to an amino acid or protein sequence may be determined over the whole sequence taught herein as the mature sequence, e.g. SEQ ID No. 7 or SEQ ID No. 8. Suitably, the degree of identity with regard to an amino acid or protein sequence may be determined over the whole sequence taught herein as SEQ ID No. 8.

In the present context, the term “query sequence” means a homologous sequence or a foreign sequence, which is aligned with a subject sequence in order to see if it falls within the scope of the present invention. Accordingly, such query sequence can for example be a prior art sequence or a third party sequence.

In one preferred embodiment, the sequences are aligned by a global alignment program and the sequence identity is calculated by identifying the number of exact matches identified by the program divided by the length of the subject sequence.

In one embodiment, the degree of sequence identity between a query sequence and a subject sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the length of the subject sequence.

In yet a further preferred embodiment, the global alignment program is selected from the group consisting of CLUSTAL and BLAST (preferably BLAST) and the sequence identity is calculated by identifying the number of exact matches identified by the program divided by the length of the subject sequence.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as omithine (hereinafter referred to as Z), diaminobutyric acid omithine (hereinafter referred to as B), norleucine omithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, ß-alanine*, L-α-amino butyric acid*, L-γ-amino butyric acid*, L-α-amino isobutyric acid*, L-ε-amino caproic acid^{#}, 7-amino heptanoic acid*, L-methlonine sulfone^{#}*, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences of the present invention.

The present invention also encompasses the use of nucleotide sequences that are complementary to the sequences presented herein, or any derivative, fragment or derivative thereof. If the sequence is complementary to a fragment thereof then that sequence can be used as a probe to identify similar coding sequences in other organisms etc.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other homologues may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of any one of the sequences in the attached sequence listings under conditions of medium to high stringency. Similar considerations apply to obtaining species homologues and allelic variants of the polypeptide or nucleotide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

Polynucleotides (nucleotide sequences) of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as DNA polynucleotides and probes according to the invention may be produced recombinantly, synthetically, or by any means available to those of skill in the art. They may also be cloned by standard techniques.

In general, primers will be produced by synthetic means, invoMng a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. The primers may be designed to contain suitable restriction enzyme recognition sites so that the amplified DNA can be cloned into a suitable cloning vector.

### Amino Acid Numbering

In the present invention, a specific numbering of amino acid residue positions in the xylanases used in the present invention may be employed. By alignment of the amino acid sequence of a sample xylanases with the xylanase of the present invention (particularly SEQ ID No. 7) it is possible to allot a number to an amino acid residue position in said sample xylanase which corresponds with the amino acid residue position or numbering of the amino acid sequence shown in SEQ ID No. 7 of the present invention.

### Hybridisation

The present invention also encompasses the use of enzymes encoded by sequences that are complementary to the nucleic acid sequences of the enzymes disclosed herein or the use of enzymes that are encoded by sequences that are capable of hybridising either to the sequences of the enzymes disclosed herein or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the sequences presented herein, or any derivative, fragment or derivative thereof.

The term “variant” also encompasses sequences that are complementary to sequences that are capable of hybridising to the nucleotide sequences presented herein.

Preferably, the term “variant” encompasses sequences that are complementary to sequences that are capable of hybridising under stringent conditions (e.g. 50° C. and 0.2×SSC {1×SSC=0.15 M NaCl, 0.015 M Na₃ citrate pH 7.0}) to the nucleotide sequences presented herein.

More preferably, the term “variant” encompasses sequences that are complementary to sequences that are capable of hybridising under high stringent conditions (e.g. 65° C. and 0.1×SSC {1×SSC=0.15 M NaCl, 0.015 M Na₃ citrate pH 7.0}) to the nucleotide sequences presented herein.

The present invention also relates to the use of nucleotide sequences that can hybridise to the nucleotide sequences of the present invention (including complementary sequences of those presented herein).

The present invention also relates to use of nucleotide sequences that are complementary to sequences that can hybridise to the nucleotide sequences of the present invention (including complementary sequences of those presented herein).

Also included within the scope of the present invention are polynucleotide sequences that are capable of hybridising to the nucleotide sequences presented herein under conditions of intermediate to maximal stringency.

In a preferred aspect, the present invention covers the use of nucleotide sequences that can hybridise to a nucleotide sequence encoding an enzyme for use in the present invention, or the complement thereof, under stringent conditions (e.g. 50° C. and 0.2×SSC).

In a more preferred aspect, the present invention covers the use of nucleotide sequences that can hybridise to a nucleotide sequence encoding an enzyme for use in the present invention, or the complement thereof, under high stringent conditions (e.g. 65° C. and 0.1×SSC).

### Expression of Enzymes

The nucleotide sequence for use in the present invention may be incorporated into a recombinant replicable vector. The vector may be used to replicate and express the nucleotide sequence, in enzyme form, in and/or from a compatible host cell.

Expression may be controlled using control sequences e.g. regulatory sequences.

The protein produced by a host recombinant cell by expression of the nucleotide sequence may be secreted or may be contained intracellularty depending on the sequence and/or the vector used. The coding sequences may be designed with signal sequences which direct secretion of the substance coding sequences through a particular prokaryotic or eukaryotic cell membrane.

### Expression Vector

The term “expression vector” means a construct capable of in vivo or in vtro expression.

Preferably, the expression vector is incorporated into the genome of a suitable host organism. The term “incorporated” preferably covers stable incorporation into the genome.

The nucleotide sequence of the present invention may be present in a vector in which the nucleotide sequence is operably linked to regulatory sequences capable of providing for the expression of the nucleotide sequence by a suitable host organism.

The vectors for use in the present invention may be transformed into a suitable host cell as described below to provide for expression of a polypeptide of the present invention.

The choice of vector e.g. a plasmid, cosmid, or phage vector will often depend on the host cell into which it is to be introduced.

The vectors for use in the present invention may contain one or more selectable marker genes—such as a gene, which confers antibiotic resistance e.g. ampicillin, kanamycin, chloramphenicol or tetracyclin resistance. Alternatively, the selection may be accomplished by co-transformation (as described in WO91/17243).

Vectors may be used in vitro, for example for the production of RNA or used to transfect, transform, transduce or infect a host cell.

Thus, in a further embodiment, the invention provides a method of making nucleotide sequences of the present invention by introducing a nucleotide sequence of the present invention into a replicable vector, introducing the vector into a compatible host cell, and growing the host cell under conditions which bring about replication of the vector.

The vector may further comprise a nucleotide sequence enabling the vector to replicate in the host cell in question. Examples of such sequences are the origins of replication of plasmids pUC19, pACYC177, pUB110, pE194, pAMB1 and plJ702.

### Regulatory Sequences

In some applications, the nucleotide sequence for use in the present invention is operably linked to a regulatory sequence which is capable of providing for the expression of the nucleotide sequence, such as by the chosen host cell. By way of example, the present invention covers a vector comprising the nucleotide sequence of the present invention operably linked to such a regulatory sequence, i.e. the vector is an expression vector.

The term “operably linked” refers to a juxtaposition wherein the components described are in a relationship permitting them to function in their intended manner. A regulatory sequence “operably linked” to a coding sequence is ligated in such a way that expression of the coding sequence is achieved under condition compatible with the control sequences.

The term “regulatory sequences” includes promoters and enhancers and other expression regulation signals.

The term “promoter” is used in the normal sense of the art, e.g. an RNA polymerase binding site.

Enhanced expression of the nucleotide sequence encoding the enzyme of the present invention may also be achieved by the selection of heterologous regulatory regions, e.g. promoter, secretion leader and terminator regions.

Preferably, the nucleotide sequence according to the present invention is operably linked to at least a promoter.

Other promoters may even be used to direct expression of the polypeptide of the present invention.

Examples of suitable promoters for directing the transcription of the nucleotide sequence in a bacterial, fungal or yeast host are well known in the art.

The promoter can additionally include features to ensure or to increase expression in a suitable host. For example, the features can be conserved regions such as a Pribnow Box or a TATA box.

### Constructs

The term “construct”—which is synonymous with terms such as “conjugate”, “cassette” and “hybrid”—includes a nucleotide sequence for use according to the present invention directly or indirectly attached to a promoter.

An example of an indirect attachment is the provision of a suitable spacer group such as an intron sequence, such as the Shi-intron or the ADH intron, intermediate the promoter and the nucleotide sequence of the present invention. The same is true for the term “fused” in relation to the present invention which includes direct or indirect attachment. In some cases, the terms do not cover the natural combination of the nucleotide sequence coding for the protein ordinarily associated with the wild type gene promoter and when they are both in their natural environment.

The construct may even contain or express a marker, which allows for the selection of the genetic construct.

For some applications, preferably the construct of the present invention comprises at least the nucleotide sequence of the present invention operably linked to a promoter.

### Host Cells

The term “host cell”—in relation to the present invention includes any cell that comprises either the nucleotide sequence or an expression vector as described above and which is used in the recombinant production of a protein having the specific properties as defined herein.

In one embodiment the organism is an expression host.

Thus, a further embodiment of the present invention provides host cells transformed or transfected with a nucleotide sequence that expresses the protein of the present invention. The cells will be chosen to be compatible with the said vector and may for example be prokaryotic (for example bacterial), fungal or yeast cells.

Examples of suitable bacterial host organisms are gram positive or gram negative bacterial species.

In one embodiment the xylanases taught herein are expressed in the expression host Trichoderma reesei.

In some embodiments the expression host for the xylanases taught herein may be one or more of the following fungal expression hosts: Fusarium spp. (such as Fusarium oxysporum); Aspergillus spp. (such as Aspergillus niger, A. oryzae, A. nidulans, or A. awamori) or Trichoderma spp. (such as T. reesei).

In some embodiments the expression host may be one or more of the following bacterial expression hosts: Streptomyces spp. or Bacillus spp. (e.g. Bacillus subtilis or B. licheniformis).

The use of suitable host cells—such as yeast and fungal host cells—may provide for posttranslational modifications (e.g. myristoylation, glycosylation, truncation, lipidation and tyrosine, serine or threonine phosphorylation) as may be needed to confer optimal biological activity on recombinant expression products of the present invention.

### Organism

The term “organism” in relation to the present invention includes any organism that could comprise the nucleotide sequence coding for the polypeptide according to the present invention and/or products obtained therefrom, and/or wherein a promoter can allow expression of the nucleotide sequence according to the present invention when present in the organism.

In one embodiment the organism is an expression host.

Suitable organisms may include a prokaryote, fungus, yeast or a plant.

The term “transgenic organism” in relation to the present invention includes any organism that comprises the nucleotide sequence coding for the polypeptide according to the present invention and/or the products obtained therefrom, and/or wherein a promoter can allow expression of the nucleotide sequence according to the present invention within the organism. Preferably the nucleotide sequence is incorporated in the genome of the organism.

The term “transgenic organism” does not cover native nucleotide coding sequences in their natural environment when they are under the control of their native promoter which is also in its natural environment.

Therefore, the transgenic organism of the present invention includes an organism comprising any one of, or combinations of, the nucleotide sequence coding for the polypeptide according to the present invention, constructs according to the present invention, vectors according to the present invention, plasmids according to the present invention, cells according to the present invention, tissues according to the present invention, or the products thereof.

For example the transgenic organism may also comprise the nucleotide sequence coding for the polypeptide of the present invention under the control of a heterologous promoter.

### Transformation of Host Cells/Organism

As indicated earlier, the host organism can be a prokaryotic or a eukaryotic organism. Examples of suitable prokaryotic hosts include E. coli, Streptomyces spp. and Bacillus spp., e.g. Bacillus subtilis.

Teachings on the transformation of prokaryotic hosts is well documented in the art, for example see Sambrook et al (Molecular Cloning: A Laboratory Manual, 2nd edition, 1989, Cold Spring Harbor Laboratory Press). If a prokaryotic host is used then the nucleotide sequence may need to be suitably modified before transformation—such as by removal of introns.

Filamentous fungi cells may be transformed using various methods known in the art—such as a process involving protoplast formation and transformation of the protoplasts followed by regeneration of the cell wall in a manner known. The use of Aspergillus as a host microorganism is described in EP 0 238 023.

Transformation of prokaryotes, fungi and yeasts are generally well known to one skilled in the art.

A host organism may be a fungus—such as a mould. Examples of suitable such hosts include any member belonging to the genera Trichoderma (e.g. T. reesei), Thermomyces, Acremonium, Fusarium, Aspergillus, Penicillium, Mucor, Neurospora and the like.

In one embodiment, the host organism may be a fungus. In one preferred embodiment the host organism belongs to the genus Trichoderma, e.g. T. reese).

### Culturing and Production

Host cells transformed with the nucleotide sequence for use in the present invention may be cultured under conditions conducive to the production of the encoded polypeptide and which facilitate recovery of the polypeptide from the cells and/or culture medium.

The medium used to cultivate the cells may be any conventional medium suitable for growing the host cell in questions and obtaining expression of the polypeptide.

The protein produced by a recombinant cell may be displayed on the surface of the cell.

The protein may be secreted from the host cells and may conveniently be recovered from the culture medium using well-known procedures.

### Secretion

Often, it is desirable for the protein to be secreted from the expression host into the culture medium from where the protein may be more easily recovered. According to the present invention, the secretion leader sequence may be selected on the basis of the desired expression host. Hybrid signal sequences may also be used with the context of the present invention.

### Large Scale Application

In one preferred embodiment of the present invention, the amino acid sequence is used for large scale applications.

Preferably the amino acid sequence is produced in a quantity of from 1 g per litre to about 2 g per litre of the total cell culture volume after cultivation of the host organism.

Preferably the amino acid sequence is produced in a quantity of from 100 mg per litre to about 900 mg per litre of the total cell culture volume after cultivation of the host organism.

Preferably the amino acid sequence is produced in a quantity of from 250 mg per litre to about 500 mg per litre of the total cell culture volume after cultivation of the host organism.

### General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John. Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

The invention also relates to the following aspects as defined in the following numbered paragraphs:
1. A method of preparing a corn based product said method comprising contacting a plant composition comprising (consisting of or consisting essentially of) corn or a corn by-product or a combination thereof with a xylanase comprising a polypeptide as set forth in SEQ ID No. 6 or SEQ ID No. 7 or SEQ ID No. 8; or a variant, homologue or derivative thereof having at least 85% identity with SEQ ID No. 6 or SEQ ID No. 7 or SEQ ID No. 8;
   or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% identity with SEQ ID No. 1, SEQ ID No. 2 or SEQ ID No. 3.
2. A method according to paragraph 1 wherein the method further comprises the addition of one or more additional plant materials.
3. A method according to paragraph 1 or paragraph 2 wherein the plant composition is contacted with the xylanase by admbdng the plant composition and the xylanase, spraying the xylanase onto the plant composition or dipping the plant composition into a preparation comprising the xylanase.
4. A method according to any one of the preceding paragraphs wherein the plant composition comprises a high fibre feed material.
5. A method according to any one of the preceding paragraphs wherein the corn by-product is corn gluten meal or corn Distillers Dried Grain Solubles (DDGS).
6. A method according to the preceding paragraphs wherein the corn based product is a compound feed, a compound feed component, a premix of a compound feed, a fodder, a fodder component, or a premix of a fodder.
7. A method according to any one of the preceding paragraphs, which method further comprises the step of forming the corn based product or plant composition into a meal, a pellet, a nut, a cake or a crumble.
8. A method according to any of the preceding paragraphs wherein the xylanase is used in combination with one or more of the enzymes selected from the group consisting of a protease (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)) and/or an amylase (including α-amylases (E.C. 3.2.1.1), G4-forming amylases (E.C. 3.2.1.60), β-amylases (E.C. 3.2.1.2) and γ-amylases (E.C. 3.2.1.3).
9. A method according to any of the preceding paragraphs wherein the xylanase is used in combination with a protease (e.g. subtilisin (E.C. 3.4.21.62)) and an amylase (e.g. α-amylases (E.C. 3.2.1.1)).
10. A method according to any of the preceding paragraphs wherein the xylanase is used in combination with a β-glucanase, e.g. an endo-1,3(4)-β-glucanases (E.C. 3.2.1.6).
11. A corn based product prepared by the method of any one of paragraphs 1 to 10.
12. A corn based product comprising corn and/or a corn by product and xylanase comprising:
   i) a polypeptide as set forth in SEQ ID No. 6 or SEQ ID No. 7 or SEQ ID No. 8; or a variant, fragment, homologue, fragments or derivative thereof having at least 85% identity with SEQ ID No. 6 or SEQ ID No. 7 or SEQ ID No. 8; or
   ii) a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or
   iii) a nucleotide sequence which has at least 80% identity with SEQ ID No. 1, SEQ ID No. 2 or SEQ ID No. 3.
13. A corn based product according to paragraph 11 or paragraph 12 wherein the corn based product is a corn based feed product
14.A corn based product according to paragraph 12 or paragraph 13 which further comprises one or more of the enzymes selected from the group consisting of a protease (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)) and/or an amylase (including α-amylases (E.C. 3.2.1.1), G4-forming amylases (E.C. 3.2.1.60), β-amylases (E.C. 3.2.1.2) and γ-amylases (E.C. 3.2.1.3).
15. A corn based product according to any one of paragraphs 12 to 14 which further comprises an amylase (e.g. α-amylases (E.C. 3.2.1.1)) and a protease (e.g. subtilisin (E.C. 3.4.21.62)).
16. A corn based product according to any one of paragraphs 12 to 15 which further comprises a β-glucanase, e.g. an endo-1,3(4)-β-glucanases (E.C. 3.2.1.6).
17. A method of preparing a feed additive composition, comprising admixing a xylanase comprising a polypeptide as set forth in SEQ ID No. 6 or SEQ ID No. 7 or SEQ ID No. 8; or a variant, fragment, homologue or derivative thereof having at least 85% identity with SEQ ID No. 6 or SEQ ID No. 7 or SEQ ID No. 8; or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% identity with SEQ ID No. 1, SEQ ID No. 2 or SEQ ID No. 3, with a feed acceptable carrier, diluent or excipient, and (optionally) packaging.
18. A method according to paragraph 17 wherein the xylanase is admixed with one or more of the enzymes selected from the group consisting of a protease (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)) and/or an amylase (including α-amylases (E.C. 3.2.1.1), G4-forming amylases (E.C. 3.2.1.60), β-amylases (E.C. 3.2.1.2) and γ-amylases (E.C. 3.2.1.3).
19. A method according to paragraph 17 or paragraph 18 wherein the xylanase is admixed with an amylase (e.g. α-amylases (E.C. 3.2.1.1)) and a protease (e.g. subtilisin (E.C. 3.4.21.62)).
20. A method according to any one of paragraphs 17 to 19 wherein the xylanase is admixed a β-glucanase, e.g. an endo-1,3(4)-β-glucanases (E.C. 3.2.1.6).
21. A feed additive composition comprising a xylanase comprising a polypeptide as set forth in SEQ ID No. 6 or SEQ ID No. 7 or SEQ ID No. 8; or a variant, fragment, homologue or derivative thereof having at least 85% identity with SEQ ID No. 6 or SEQ ID No. 7 or SEQ ID No. 8; or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% identity with SEQ ID No. 1, SEQ ID No. 2 or SEQ ID No. 3 and a feed acceptable carrier, diluent or excipient and optionally packaged.
22. A premix comprising a feed additive composition according to paragraph 21 or a xylanase comprising a polypeptide as set forth in SEQ ID No. 6 or SEQ ID No. 7 or SEQ ID No. 8; or a variant, fragment, homologue or derivative thereof having at least 85% identity with SEQ ID No. 6 or SEQ ID No. 7 or SEQ ID No. 8; or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% identity with SEQ ID No. 1, SEQ ID No. 2 or SEQ ID No. 3; in combination with at least one mineral and/or at least one vitamin.
23. A feed additive composition according to paragraph 21 or a premix according to paragraph 22 wherein the feed additive composition or premix is formulated as a dry powder or granules (preferably TPT granules).
24. A feed additive composition according to paragraph 21 or paragraph 23 or a premix according to paragraph 22 or paragraph 23 which further comprises one or more of the enzymes selected from the group consisting of a protease (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)) and/or an amylase (including α-amylases (E.C. 3.2.1.1), G4-forming amylases (E.C. 3.2.1.60), 3-amylases (E.C. 3.2.1.2) and -amylases (E.C. 3.2.1.3).
25. A feed additive composition according to any one of paragraph 21 or paragraphs 23-24 or a premix according to any one of paragraphs 22 to 24 which further comprises an amylase (e.g. α-amylases (E.C. 3.2.1.1)) and a protease (e.g. subtilisin (E.C. 3.4.21.62)).
26. A feed additive composition according to any one of paragraph 21 or paragraphs 23-25 or a premix according to any one of paragraphs 22 to 25 which further comprises a β-glucanase, e.g. an endo-1,3(4)-β-glucanases (E.C. 3.2.1.6).
27. A method of improving the performance of a subject or improving digestibility (e.g. nutrient digestibility) or improving feed efficiency in a subject comprising administering:
   (i) a corn based product prepared in accordance with any one of paragraphs 1 to 10 or according to any one of paragraphs 11-16; or
   (ii) a feed additive composition according to any one of paragraphs 21 or 23-26 or a premix according to any one of paragraphs 22 or 23-26; or
   (iii) a xylanase comprising a polypeptide as set forth in SEQ ID No. 6 or SEQ ID No. 7 or SEQ ID No. 8; or a variant, fragment, homologue or derivative thereof having at least 85% identity with SEQ ID No. 6 or SEQ ID No. 7 or SEQ ID No. 8;
      or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% identity with SEQ ID No. 1, SEQ ID No. 2 or SEQ ID No. 3;
   wherein in (ii) and (iii) the subject is further administered a plant composition comprising corn or a corn by-product.
28. Use of a corn based product in accordance with any one of paragraphs 11 to 16 or a portion thereof, or a feed additive composition according to any one of paragraphs 21 or 23-26 or a premix according to any one of paragraphs 22 or 23-26, or a xylanase comprising a polypeptide as set forth in SEQ ID No. 6 or SEQ ID No. 7 or SEQ ID No. 8; or a variant, fragment, homologue or derivative thereof having at least 85% identity with SEQ ID No. 6 or SEQ ID No. 7 or SEQ ID No. 8;
   or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% identity with SEQ ID No. 1, SEQ ID No. 2 or SEQ ID No. 3; to improve the performance of a subject or improve digestibility (e.g. nutrient digestibility) in a subject or improve feed efficiency in a subject, particularly in relation to corn-based feed products.
29. A kit comprising a feed additive composition according to any one of paragraphs 21 or 23-26 or a premix according to any one of paragraphs 22 or 23-26 and instructions for administration with a corn-based feed product.
30. A use of a xylanase comprising a polypeptide as set forth in SEQ ID No. 6 or SEQ ID No. 7 or SEQ ID No. 8; or a variant, fragment, homologue or derivative thereof having at least 85% (suitably at least 90% or at least 95%) identity with SEQ ID No. 6 or SEQ ID No. 7 or SEQ ID No. 8; or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% (suitably at least 85% or at least 90% or at least 95%) identity with SEQ ID No. 1, SEQ ID No. 2 or SEQ ID No. 3, in the production of a fermented beverage, such as a beer.
31. A method of producing a fermented beverage comprising the step of contacting a mash and/or a wort with a xylanase comprising a polypeptide as set forth in SEQ ID No. 6 or SEQ ID No. 7 or SEQ ID No. 8; or a variant, fragment, homologue or derivative thereof having at least 85% identity with SEQ ID No. 6 or SEQ ID No. 7 or SEQ ID No. 8; or encoded by a nucleotide sequence shown herein as SEQ ID No. 1, SEQ ID No. 2 or SEQ ID No. 3, or a nucleotide sequence which can hybridize to the complement of SEQ ID No. 1, SEQ ID No. 2 or SEQ ID No. 3 under high stringency conditions, or a nucleotide sequence which has at least 80% identity with SEQ ID No. 1, SEQ ID No. 2 or SEQ ID No. 3.
32. A method of producing a fermented beverage according to paragraph 31, wherein the method comprises the steps of (a) preparing a mash, (b) filtering the mash to obtain a wort, and (c) fermenting the wort to obtain a fermented beverage, such as a beer, and wherein said xylanase is added to: (i) the mash of step (a) and/or (ii) the wort of step (b) and/or (iii) the wort of step (c).
33. A fermented beverage, such as a beer, produced by a method of paragraph 31 or paragraph 32.
34. A method, use, kit, feed product or feed additive substantially as disclosed herein with reference to the Figures and Examples.

The invention will now be described, by way of example only, with reference to the following Figures and Examples.

### EXAMPLES

### Example 1

Cloning of the Aspergillus clavatus Xylanase AclXyn5

The entire genomic sequence data of Aspergillus clavatus is available online (http//www.broadinstitute.or/annotation/aenome/aspemillus_group/GeneDetails.html?sp=S 7000001156845959) One of the genes (ACLA_063140) identified in Aspergillus clavatus encodes a glycosyl hydrolase with homology to xylanases of various other fungi as determined from a BLAST search (Altschul et al., J Mol Biol, 215: 403-410, 1990). The nucleotide sequence of this gene, called AclXyn5 gene, is depicted as SEQ ID NO.1. The protein encoded by the AclXyn5 gene is depicted as SEQ ID NO. 6, and has received the accession number A1CCU0 in Uniprot database. Genomic DNA of Aspergillus clavatus was used for amplifying the AclXyn5 gene for expression. The protein product of the AclXDyn5 gene belongs to Glycosyl hydrolase family 11 based on the PFAM search (http://pfam.sanger.ac.uk/). At the N-terminus, AclXyn5 protein has an 18 amino acid signal peptide predicted by SignalP-NN (Emanuelsson et al., Nature Protocols, 2:953-971, 2007). This indicates that AclXyn5 is a secreted glycosyl hydrolase.

### Example 2

### Expression of AclXyn5 Protein

The AclXyn5 gene was amplified from genomic DNA of Aspergillus clavatus using the following primers: Primer 1(Not I) 5′-ccgcggccgcaccATGGTGTCGTTCAAGTATCTTTTCCT-3′ (SEQ ID NO: 4), and Primer 2 (Asc I) 5′-ccggcgcgcccttaTTAATAGACAGTAATGGAGGAGGAAC-3′ (SEQ ID NO: 5). After digestion with Not I and Asc I enzymes, the PCR product was cloned into pTrex3gM expression vector (described in US 2011/0136197 A1) digested with the same restriction enzymes, and the resulting plasmid was designated pZZH159. The map of plasmid pZZH159 is provided in FIG. 7. The sequence of the AclXDyn5 gene was confirmed by DNA sequencing. The plasmid pZZH159 was transformed into a quad deleted Trichoderma reesei strain (described in WO 05/001036) using biolistic method (Te'o V S et al., J Microbiol Methods, 51:393-9, 2002).

Following sequence confirmation, protoplasts of a quad deleted T. reesei strain (described in WO 05/001036) were transformed with the expression plasmid pTT-Ate CA1 using the PEG protoplast method (Penttila et al, Gene, 61:155-164, 1987). For protoplast preparation, spores were grown for about 10 hours at 24° C. in Trichoderma Minimal Medium MM (20 g/L glucose, 15 g/L KH₂PO₄, pH 4.5, 5 g/L (NH₄)2SO₄, 0.6 g/L MgSO₄×7H₂O, 0.6 g/L CaCl₂×2H₂O, 1 ml of 1000× T. reesei Trace elements solution (175 g/L Citric Acid anhydrous, 200 g/L FeSO₄×7H₂O, 16 g/L ZnSO₄×7H₂O, 3.2 g/L CuSO₄, 1.4 g/L MnSO₄xH2O, and 0.8 g/L Boric Acid). Germinating spores were harvested by centrifugation and treated with 30 mg/mL Vinoflow FCE (Novozymes, AG Switzerland) solution for from 7 hours to overnight at 30° C. at 100 rpm to lyse the fungal cell walls. Protoplasts were washed in 0.1 M Tris HCl buffer (pH 7) containing 0.6 M sorbitol and resuspended in 10 mM Tris HCl buffer (pH 7.5) containing 1.2 M sorbitol and 10 mM calcium chloride. For PEG transformation, approximately 1 μg of DNA and 1-5×10⁷ protoplasts in a total volume of 200 μl were treated with 2 ml of 25% PEG solution, diluted with 2 volumes of 1.2 M sorbitol/10 mM Tris, pH 7.5/10 mM CaCl₂ solution.

Transformants were selected on a medium containing acetamide as a sole source of nitrogen (acetamide 0.6 g/L; cesium chloride 1.68 g/L; glucose 20 g/L; potassium dihydrogen phosphate 15 g/L; magnesium sulfate heptahydrate 0.6 g/L; calcium chloride dihydrate 0.6 g/L; iron (II) sulfate 5 mg/L; zinc sulfate 1.4 mg/L; cobalt (II) chloride 1 mg/L; manganese (II) sulfate 1.6 mg/L; agar 20 g/L; pH 4.25). Transformed colonies (about 50-100) appeared in about 1 week. After growth on acetamide plates, the spores were collected and reselected on acetamide plates. After 5 days, the spores were collected using 10% glycerol, and 1×10⁸ spores were inoculated in a 250 ml shake flask with 30 ml Glucose/Sophorose defined medium for protein expression. Protein expression was confirmed by SDS-PAGE. The spore suspension was subsequently grown in a 7 L fermentor in a defined medium containing 60% glucose-sophorose feed. Glucose/Sophorose defined medium (per liter) consists of (NH₄)2SO₄ 5 g, PIPPS buffer 33 g, Casamino Acids 9 g, KH₂PO₄ 4.5 g, CaCl₂ (anhydrous) 1 g, MgSO₄.7H₂O 1 g, pH to 5.5 adjusted with 50% NaOH with Milli-Q H₂O to bring to 966.5 mL. After sterilization, the following were added: 26 mL 60% Glucose/Sophrose, and 400× T. reesei Trace Metals 2.5 mL.

AclXyn5 protein was purified from concentrated 7 L fermentor culture supernatant using two chromatography columns. Concentrated culture supernatant buffered in 20 mM sodium phosphate buffer pH 6.0 containing 1 M ammonium sulfate was loaded on a hydrophobic interaction chromatography column (Sepharose Butyl FF, XK 26/10). The protein was eluted from the column using a linear gradient of equilibration/wash buffer to 20 mM sodium phosphate buffer pH 6.0. The fraction containing the AclXyn5 protein was loaded onto a gel filtration column (HiLoad Superdex 75 pg 26/60), and the mobile phase used was 20 mM sodium phosphate, pH 7.0, containing 0.15 M NaCl. The purified protein was concentrated using a 3K Amicon Ultra-15 device and the concentrated protein fraction was used in further studies.

The nucleotide sequence of AclXDyn5 gene from expression plasmid pZZH159 is set forth as SEQ ID NO:1. The signal sequence is shown in bold, and the predicted intron is shown in italics and lowercase.

The amino acid sequence of AclXyn5 protein expressed from plasmid pZZH159 is set forth as SEQ ID NO:6. The signal sequence predicted by SignalP-NN software is shown in italics. The amino acid sequence for the mature form of AclXyn5 protein as predicted by SignalP-NN software is set forth as SEQ ID NO:7. The amino acid sequence of a further processed mature form of the AclXyn5 protein is set forth as SEQ ID NO:8 which could arise from posttranslational processing, e.g. KexII N-terminal processing.

### Example 3

### Xylanase Activity of AclXyn5

AclXyn5 belongs to the glycosyl hydrolase family 11 (based on the CAZy numbering system). The beta 1-4 xylanase activity of AclXyn5 was measured using 1% xylan from birch wood (Sigma 95588) or 1% arabinoxylan from wheat flour (Megazyme P-WAXYM) as substrates. The assay was performed in 50 mM sodium citrate pH 5.3, 0.005% Tween-80 buffer at 50° C. for 10 minutes.

The released reducing sugar was quantified by reaction with 3, 5-Dinitrosalicylic acid and measurement of absorbance at 540 nm. The enzyme activity is quantified relative to a xylose standard curve. In this assay, one xylanase unit (U) is defined as the amount of enzyme required to generate 1 micromole of xylose reducing sugar equivalents per minute under the conditions of the assay.

### Example 4

### pH Profile of AclXyn5

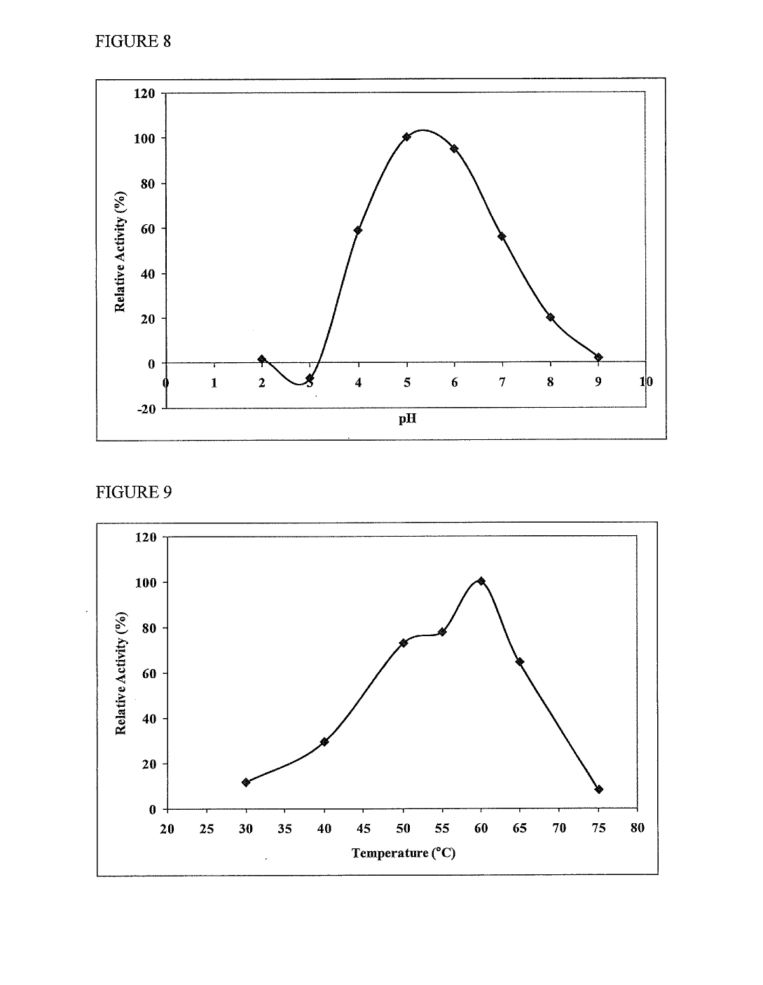

The pH profile of AclXyn5 was determined using xylan from birch wood (Sigma 95588) as substrate. The assay was performed in Sodium Citrate/Sodium Phosphate buffer solution adjusted to pH values between 2 and 9. Birchwood xylan dissolved in water to 2% solution was mixed with same volume of 50 mM Citrate/Phosphate buffer solution in a 96-well plate, and the substrate was equilibrated at 50° C. before adding enzyme. After 10 minutes, the enzyme reaction was stopped by transferring 60 microliters of reaction mixture to 100 microliters of DNS solution placed in a 96-well PCR plate. The PCR plate was heated at 95° C. for 5 minutes in the Bio-Rad DNA Engine. Then, plate was cooled to room temperature and 100 microliters from each tube was transferred to a new 96-well plate. Release of the reducing end from the substrate was quantified by measuring the optical density at 540 nm in a spectrophotometer. Enzyme activity at each pH was reported as relative activity where the activity at the pH optimum was set to 100%. The pH profile of AclXyn5 is shown in FIG. 8. AclXyn5 was found to have an optimum pH at about 5, and was found to retain greater than 70% of maximum activity between pH 4.3 and 6.6.

### Example 5

### Temperature Profile of AclXyn5

The temperature optimum of purified AclXyn5 was determined by assaying for xylanase activity at temperatures varying between 30° C. and 75° C. for 10 minutes in 50 mM sodium citrate buffer at pH 5.3. The activity was reported as relative activity where the activity at the temperature optimum was set to 100%. The temperature profile of AclXyn5 is shown in FIG. 9. AclXyn5 was found to have an optimum temperature of 60° C., and was found to retain greater than 70% of maximum activity between 49° C. and 64° C.

### Example 6

### Pentosan Solublisation (Breakdown of Insoluble Arabinoxylan (AXinsol))

The xylanase (AclXyn 5) was cloned, expressed, purified and characterised and tested against a benchmark xylanase product Econase® XT.

### 6.1 Materials and Methods

### Enzyme Samples

The xylanases used in this study are:

A GH11 xylanase from Aspergillus clavatus (designated AclXyn 5) expressed in Trichoderma reesei—the enzyme was used as a sterile filtered ferment, and the following benchmark, commercially available xylanase: Econase® XT. This benchmark enzyme was extracted from commercial dry formulated samples. The xylanase component from Econase® XT commercial dry formulated samples was extracted in a 33% (w/w) slurry using McIlvain buffer, pH 5.0. The extract was cleared using centrifugation (3000 RCF for 10 min) and filtered using a PALL Acrodisc PF syringe filter (0.8/0.2 μm Supor membrane) and subsequently heated 20 min at 70° C. After removable of precipitation by centrifugation (38 724 RCF for 15 min) the buffer was replaced by passage through a Sephadex G25 column (PD10 from Pharmacia) equilibrated with 20 mM Na Citrate, 20 mM NaCl, pH 3.4. Purification of the xylanase component was performed using Source 15S resin, followed by elution with a linear increasing salt gradient (NaCl in 20 mM Na Citrate buffer pH 3.4).

Econase XT® is an endo-1,4-xylanase (EC 3.2.1.8) produced by the strain Trichoderma reesei RF5427 (CBS 114044), available from ABVista.

Protein concentration was determined by measuring absorption at 280 nm. The extinction coefficients were estimates from the amino acid sequences. For Econase XT the absorption at 280 nm of 1 mg/ml was calculated to be 2.84 AU.

### Feed Raw Materials

The feed used in these experiments is raw material. The feeds are either corn or corn DDGS.

### Pentosan Solubilization (AXinsol Solubilisation)

The method used for pentosan solubilisation was: 100 mg of feed raw material was transferred to a 2 ml Eppendorf centrifuge tube and the precise weight recorded. 750 μL incubation buffer (200 mM HEPES, 100 mM NaCl, 2 mM CaCl, pH 6.0) and 900 μl chloramphenicol solution (40 μg/ml in incubation buffer) was added. Enzyme of choice was added to make a total volume of 1.8 mL.

Each sample was assayed in doublets and in parallel with a blank (incubation without exogenously added enzyme). The samples were Incubated on an Eppendorf thermomixer at 40° C. with shaking. After 2 or 18 hours of incubation the supernatant was filtered using 96 wells filterplates (Pall Corporation, AcroPrep 96 Filter Plate, 1.0 μm Glass, NTRL, 1 mL well). After filtration the samples were stored at 4° C. until analysis for total amount of C5 sugars, arabinose and xylose.

### Quantification of C5 Sugars (Pentosans)

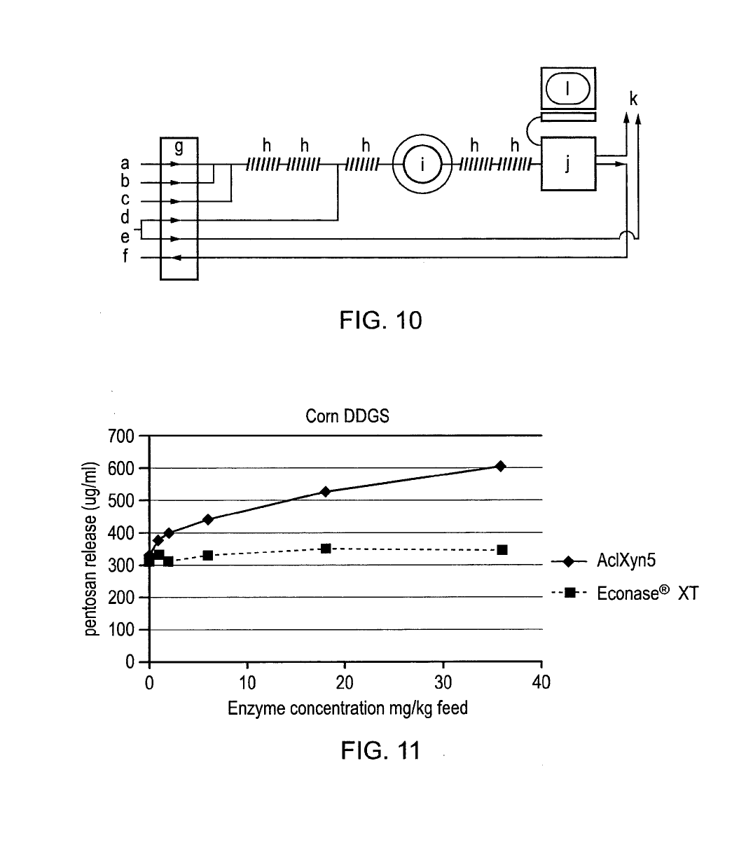

The total amount of pentoses brought into solution was measured using the method of Rouau and Surget (1994, A rapid semi-automated method of the determination of total and water-extractable pentosan in wheat flours. Carbohydrate Polymers, 24, 123-32) with a continuous flow injection apparatus (FIG. 10). The supernatants were treated with acid to hydrolyse polysaccharides to monosugars. Phloroglucinol (1,3,5-trihydroxybenzen) was added for reaction with monopentoses and monohexoses, which forms a coloured complex.

By measuring the difference in absorbance at 550 nm compared to 510 nm, the amount of pentoses in the solution was calculated using a standard curve. Unlike the pentose-phloroglucinol complex, the absorbance of the hexose-phloroglucinol complex is constant at these wavelengths. Glucose was added to the phloroglucinol solution to create a constant glucose signal and further ensure no interference from hexose sugars. The pentose concentration in the samples was determined using a xylose standard curve.

### 6.1. Results

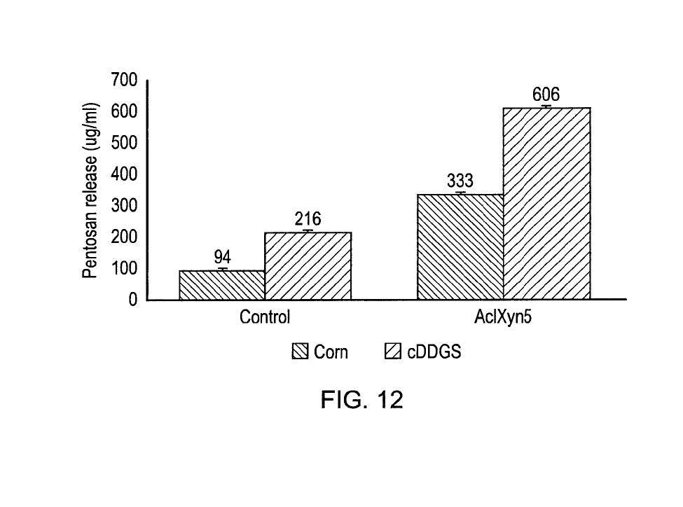

AclXyn5 performed surprisingly strongly in pentosan solubilisation of both corn and cDGGS (see FIG. 12).

It can be seen from FIG. 11 that AclXyn5 surprisingly far out-performs the commercial benchmark in pentosan solubilisation (e.g. degradation of arbinoxylan to pentosans (e.g. xylose)) in cDGGS.

### Pentosan Solublisation

Pentosan solubilisation was monitored in a dose response setup using a fibrous by-product of corn (namely cDDGS).

The results from benchmark Econase® XT and AcylXyn5 on pentosan solubilisation are shown in FIG. 11 (in corn DDGS).

FIG. 11 shows solubilisation of pentosans from cDDGS as a function of xylanase dosage. The xylanases used were the xylanase of the present invention (AclXyn 5) compared with the benchmark xylanase Econase® XT. The order of legends indicates the ranking at the highest xylanase dose (36 mg/kg feed).

Econase® XT shows no or limited effect with regard to pentosan solublisation on corn. AclXyn5 surprisingly outperforms the benchmark enzyme Econase® XT when solubilising pentosans in corn (see FIG. 11).

This indicates a clear difference in substrate specificity for AclXyn5 compared with Econase® XT. With the new xylanase (AclXyn5) having a broader substrate specificity with regard to pentosan solublisation compared with the benchmark enzyme.

### Example 7

### Xylanase (AclXyn5) in Pigs

### 7.1. Materials and Methods

This experiment is conducted to evaluate the efficacy of AclXyn5 on ileal nutrients and energy digestibility in growing pigs fed wheat/wheat bran based diets. An Animal Care and Use Committee approves the use of the pigs and relevant welfare guidelines for the Country are used. Six growing barrows (initial body weight of 30 kg) are equipped with a T-cannula in the distal ileum for the purpose of the experiment. Pigs are of the off springs of G-Performer boars that are mated to Fertilium 25 females (Genetiporc, Alexandria, Minn., USA) and are housed in individual pens (1.2×1.5 m) in an environmentally controlled room. Each pen is equipped with a feeder and a nipple drinker and has fully slatted concrete floors.

TABLE 7.1
Composition of the basal diet

| Item | Level |
|---|---|
| Hard Wheat | 56.57 |
| Wheat Bran | 5.06 |
| Wheat middlings | 19.94 |
| Soybean Meal | 13.50 |
| Soybean oil | 0.99 |
| L-Lysine HCl | 0.73 |
| DL-Methionine | 0.17 |
| L-Threonine | 0.30 |
| L-Tryptophan | 0.01 |
| Digestibility marker (celite) | 0.30 |
| Salt | 0.47 |
| Limestone | 1.38 |
| Monocalcium Phosphate | 0.08 |
| Vitamins/Trace minerals premix¹ | 0.50 |
| Calculated provisions | |
| Crude protein, % | 19.11 |
| Net energy, MJ/kg | 8.88 |
| SID Lysine g/NE MJ | 1.31 |
| SID Lysine, % | 1.16 |
| SID Methionine, % | 0.35 |
| Neutral detergent fibre, % | 17.93 |
| Calcium, % | 0.68 |
| Available phosphorous, % | 0.22 |

¹Provided the following quantities of vitamins and trace minerals per kilogram of complete diet: Vitamin A as retinyl acetate, 11,128 IU; vitamin D₃ as cholecalciferol, 2,204 IU; vitamin E as DL-alpha tocopheryl acetate, 66 IU; vitamin K as menadione nicotinamide bisulfite, 1.42 mg; thiamin as thiamine mononitrate, 0.24 mg; riboflavin, 6.58 mg; pyridoxine as pyridoxine hydrochloride, 0.24 mg; vitamin B₁₂, 0.03 mg; D-pantothenic acid as D-calcium pantothenate, 23.5 mg; niacin as nicotinamide and nicotinic acid, 44 mg; folic acid, 1.58 mg; biotin, 0.44 mg; Cu, 10 mg as copper sulfate; Fe, 125 mg as iron sulfate; I, 1.26 mg as potassium iodate; Mn, 60 mg as manganese sulfate; Se, 0.3 mg as sodium selenite; and Zn, 100 mg as zinc oxide.

TABLE 7.2
Treatments identification

| | | Phytase¹ | | |
|---|---|---|---|---|
| Diet | Treatment ID | (FTU/kg of feed) | Xylanase | |
| Control | 1 | 500 FTU | 0 | |
| Control + AclXyn5 | 2 | 500 FTU | 4000 | U/kg |
| Control + Commercial | 3 | 500 FTU | 75 | ppm |
| xylanase² | | | | |

¹Phytase from Danisco Animal Nutrition
²Econase ® XT from AB Vista

A basal diet is formulated to meet the NRC nutrients recommendations for swine (NRC, 1998; Table 7.1). One batch of the basal diet is manufactured and split into three portions and each portion subsequentlymixed with additives identified in Table 7.2. The experiment is designed and conducted according to a 4×4 Latin square design with 2 added columns to give 6 replicates per diet. All pigs are fed at a level of 3 times their maintenance energy requirement (106 kcal ME per kg^{0.75}; NRC, 1998), and provided at 0800 and 1700 h. Animals have free access to water through a bowl-type drinker. Pig weights are recorded at the beginning and at the end of each period and the amount of feed supplied each day are recorded. Each experimental period lasted for 7 d. The initial 5 days of each period are considered an adaptation period to the diet. Heal digesta are collected for 8 h on d 6 and 7 using standard operating procedures. In brief, a plastic bag is attached to the cannula barrel and digesta flowing into the bag is collected. Bags are removed whenever they are filled with digesta—or at least once every 30 min and are immediately frozen at −20° C. On the completion of one experimental period, animals are deprived of feed overnight and the following morning, a new experimental diet is offered.

At the end of the experiment, ileal samples are thawed, mixed within animal and diet, and a sub-sample is collected for chemical analysis. A sample of basal diet is also collected and analyzed. Digesta samples are lyophilized and finely ground prior to chemical analysis. All samples are analyzed for dry matter, Titanium, gross energy, crude protein, fat and neutral detergent fibre according to standard procedures (AOAC, 2005). The values for apparent ileal digestibility of energy and nutrients are calculated as described previously (Stein et al., 2007 Livestock Science, 2007 vol. 109, issue 1 part 3 p 282-285 and J ANIM SCI January 2007 vol. 85 no. 1 172-180). Data were analyzed using the MIXED procedures of SAS.

### 7.1 Results and Discussion

Pigs fed AclXyn5 had significantly higher (P<0.05) apparent ileal digestibility of dry matter and fat than commercial xylanase fed pigs (Table 7.3); AclXyn5 also showed numerically higher dry matter and fat digestibility than the control. Subsequently pigs fed AclXyn5 extracted 69 and 182 kcal extra (Table 7.3) energy compared to pigs fed the control and commercial xylanase, respectively.

TABLE 7.3
Effect of new xylanase on ileal nutrients digestibility (%) and
energy utilization (kcal/kg) in growing pigs fed wheat based diet

| | Dry matter | Crude protein | Fat | Energy |
|---|---|---|---|---|
| Control | 68.0ab | 74.7 | 62.2ab | 3,168ab |
| AclXyn5 | 69.4a | 76.5 | 66.7a | 3,237a |
| Commercial xylanase | 65.8b | 74.6 | 56.3b | 3,055b |
| (Econase ® XT) | | | | |
| SEM | 1.35 | 0.99 | 2.47 | 50.9 |

Within a column, means with different letter signs are significantly different; P < 0.05.

### Example 8

### 8.1 Materials and Methods

The efficacy of AclXyn5 on ileal and total tract nutrients and energy digestibility in growing pigs fed com-corn DDGS and wheat/wheat bran based diets. The protocol for the experiment is reviewed and approved by an Institutional Animal Care and Use Committee and relevant welfare guidelines for the Country are used. A total of 24 barrows ([♀Yorkshire×Landrace]×♂Duroc; initial body weight of 30 kg) will be equipped with a T-cannula in the distal ileum for the purpose of the experiment. The pigs will be individually housed in metabolism crates that a smooth transparent plastic sides and plastic-covered expanded metal sheet flooring in a temperature-controlled room (22±2° C.).

TABLE 8.1
Composition of the basal diets

| Item | Corn based | Wheat based |
|---|---|---|
| Corn | 41.44 | |
| US corn DDGS | 40.00 | |
| Hard Wheat | | 56.57 |
| Wheat Bran | | 5.06 |
| Wheat middlings | | 19.94 |
| Soybean Meal | 15.00 | 13.50 |
| Tallow | | 0.99 |
| L-Lysine HCl | 0.75 | 0.73 |
| DL-Methionine | 0.10 | 0.17 |
| L-Threonine | 0.25 | 0.30 |
| L-Tryptophan | 0.06 | 0.01 |
| Digestibility marker (celite) | 0.30 | 0.30 |
| Salt | 0.30 | 0.47 |
| Limestone | 1.30 | 1.38 |
| Monocalcium Phosphate | 0.00 | 0.08 |
| Vitamins/Trace minerals premix¹ | 0.50 | 0.50 |
| Calculated provisions | | |
| Crude protein, % | 21.44 | 19.11 |
| Net energy, MJ/kg | 8.88 | 8.88 |
| SID Lysine g/NE MJ | 1.31 | 1.31 |
| SID Lysine, % | 1.16 | 1.16 |
| SID Methionine, % | 0.35 | 0.35 |
| Neutral detergent fibre, % | 20.63 | 17.93 |
| Calcium, % | 0.67 | 0.68 |
| Available phosphorous, % | 0.22 | 0.22 |

¹The vitamin and trace mineral premix provided the following (per kg of diet): vitamin A, 11,000 IU; vitamin D3, 2,756 IU; vitamin E, 55 IU; vitamin B12, 55 μg; riboflavin, 16,000 mg; pantothenic acid, 44.1 mg; niacin, 82.7 mg; Zn, 150 mg; Fe, 175 mg; Mn, 60 mg; Cu, 17.5 mg; I, 2 mg; and Se, 0.3 mg

Respective basal diets are formulated to meet the NRC nutrients recommendations for swine (NRC, 1998 Table 8.1). In each experiment, one batch of the basal diet are manufactured and split into four portions and each portion subsequently mixed with additives identified in Table 8.2.

TABLE 8.2
Treatments identification

| | | Phytase¹ | | |
|---|---|---|---|---|
| Diet | Treatment ID | (FTU/kg of feed) | Xylanase | |
| Control | 1 | 500 FTU | 0 | |
| Control + AclXyn5 | 2 | 500 FTU | 4000 | U/kg |
| Control + Commercial | 3 | 500 FTU | 75 | ppm |
| xylanase² | | | | |

¹Phytase from Danisco Animal Nutrition
²Econase ® XT from AB Vista

The experiment is designed and conducted as two period cross-over design in which all corn diets are run in period one and all wheat diets run in period two. Within a period, the 4 treatments are allocated to pigs in a completely randomized design to give 6 replicates per treatment. Pigs are fed a common commercial diet for a week before commencement of the second period. The pigs are fed their respective diets in two equal portions at 0830 and 1630. Daily feed allowance is based on the pig's BW at the beginning of the period and is calculated to supply 2.6 times the estimated maintenance requirements. Each experimental period lasts for 14 d: d 7 for adaptation, d 8 and 9 for grab fecal collection and d 10 and 11 for ileal digesta collection to examine coefficient of apparent ileal and total tract digestibility of N, DM, energy and crude fat. Pigs are allowed free accessible to water from nipple drinkers located in each pen at all times. Digesta flow measurements and blood samples collection are conducted from d 12 to 14. Data is analysed using GLM procedures of SAS. Statistical significance will be accepted at P<0.05.

### 8.2 Results and Discussion

Preliminary results indicate that AclXyn5 outperforms the commercial benchmark xylanases in both corn and wheat based diets.

### Example 9

### AclXyn5 in Animal Feed—Poultry

### 9.1 Materials and Methods

An experiment is conducted to evaluate the efficacy of AclXyn5 on growth performance of broiler chickens fed corn/corn DDGS based diets and wheat/wheat bran based diets. The experimental procedures are approved by an Animal Ethics Committee and, complied with relevant welfare guidelines for the Country. A two-phase feeding programme (starter and finisher) is used (Table 9.1). The starter and finisher diets are offered from d 0 to 21 and 22 to 42, respectively.

TABLE 9.1
Composition of the basal diets¹

| | Starter, d 0-21 | | Finisher, d 22-42 | |
|---|---|---|---|---|
| | Corn | Wheat | Corn | Wheat |
| Corn | 57.39 | — | 58.50 | — |
| Corn DDGS | 11.00 | — | 15.00 | — |
| Wheat | — | 60.17 | — | 63.30 |
| Wheat bran | — | 9.00 | — | 13.00 |
| Soybean meal, 45% | 26.50 | 22.34 | 19.00 | 14.00 |
| Tallow | 1.75 | 4.85 | 3.20 | 5.95 |
| Vitamin-mineral premix² | 0.33 | 0.33 | 0.33 | 0.33 |
| Sodium bicarbonate | 0.20 | 0.22 | 0.20 | 0.29 |
| Salt | 0.38 | 0.38 | 0.34 | 0.35 |
| Monocalcium phosphate | 0.35 | 0.37 | 0.13 | 0.20 |
| Limestone | 1.700 | 1.69 | 1.70 | 1.65 |
| L-Lysine-HCl | 0.135 | 0.25 | 0.20 | 0.34 |
| DL-methionine | 0.185 | 0.23 | 0.13 | 0.19 |
| L-threonine | 0.100 | 0.19 | 0.09 | 0.22 |
| Calculated provisions | | | | |
| Crude protein | 21.0 | 21.1 | 18.6 | 18.2 |
| ME (MJ/kg) | 12.5 | 12.2 | 12.9 | 12.5 |
| Calcium | 0.81 | 0.80 | 0.75 | 0.73 |
| Available Phosphorous | 0.25 | 0.25 | 0.21 | 0.21 |
| Sodium | 0.23 | 0.22 | 0.23 | 0.22 |
| Digestible Lysine | 1.01 | 0.99 | 0.90 | 0.87 |
| Digestible Methionine | 0.47 | 0.47 | 0.39 | 0.39 |
| Digestible Threonine | 0.74 | 0.74 | 0.64 | 0.66 |
| Digestible Tryptophan | 0.19 | 0.21 | 0.16 | 0.17 |

¹A commercial phytase from Danisco Animal Nutrition top dressed to supply 500 FTU/kg of final feed
²Supplied per kilogram of diet: antioxidant, 100 mg; biotin, 0.2 mg; calcium pantothenate, 12.8 mg; cholecalciferol, 60 μg; cyanocobalamin, 0.017 mg; folic acid, 5.2 mg; menadione, 4 mg; niacin, 35 mg; pyridoxine, 10 mg; trans-retinol, 3.33 mg; riboflavin, 12 mg; thiamine, 3.0 mg; dl-α-tocopheryl acetate, 60 mg; choline chloride, 638 mg; Co, 0.3 mg; Cu, 3.0 mg; Fe, 25 mg; I, 1 mg; Mn, 125 mg; Mo, 0.5 mg; Se, 200 μg; Zn, 60 mg.

Two basal diets, one based on wheat/wheat bran and soybean meal, and the other based on corn/corn DDGS and soybean meal, are formulated to meet or exceed the recommended requirements for nutrients, except AME, for broilers (Table 9.1). From each basal diet, three experimental diets are developed to constitute control, AclXyn5, a commercial xylanase as identified in Table 9.2.

TABLE 9.2
Treatments identification

| | | Phytase¹ | | |
|---|---|---|---|---|
| Diet | Treatment ID | (FTU/kg of feed) | Xylanase | |
| Control | 1 | 500 FTU | 0 | |
| Control + AclXyn5 | 2 | 500 FTU | 4000 | U/kg |
| Control + Commercial | 3 | 500 FTU | 75 | ppm |
| xylanase ² | | | | |

¹Phytase from Danisco Animal Nutrition
² Econase ® XT from AB Vista

Male broiler (Ross 308) chicks are obtained as day-olds from a commercial hatchery. The chicks are individually weighed and allocated to 72 brooder cages (8 chicks per cage) and the 8 dietary treatments randomly assigned to eight cages each. On day 12, the birds are transferred to grower cages. The space allocation per bird in brooder and grower cages is 530 and 640 cm², respectively. The brooder and grower cages are housed in environmentally controlled rooms. The temperature is maintained at 31° C. in the first week and then gradually reduced to 22° C. by the end of third week. The birds receive 20 hours fluorescent illumination and, are allowed free access to the diets and water. Body weights and feed intake are recorded at weekly intervals throughout the 42-day experimental period. Mortality is recorded daily. Any bird that died is weighed and the weight is used to adjust FCR. Feed conversion ratios are calculated by dividing total feed intake by weight gain of live plus dead birds. Data are analysed as a two-way factorial arrangement of treatments using the General Linear Models procedure of SAS (2004).

### 9.2 Results and Discussion

Compared with the control and commercial xylanase 2, AclXyn5 improves gain and FCR in both corn and wheat based diets (Table 9.3). This demonstrates the efficaciousness of AclXyn5 in mitigating the negative effects of the insoluble and soluble fibrous fractions in cereal ingredients that may limit poultry performance and improving energy utilization.

TABLE 9.3
Effect of new xylanase on growth performance of broiler
chickens fed corn-corn DDGS and wheat-wheat bran based diets

| | | Initial | Final | | Body | Feed |
|---|---|---|---|---|---|---|
| | | body | body | Feed | weight | conversion |
| Treatments | | weight, g | weight, g | intake, g | gain, g | ratio, g/g |
| Grain | Xylanase | | | | | |
| Corn | Control | 38.3 | 2151 | 3738 | 2113 | 1.81 |
| Corn | AclXyn5 | 38.3 | 2495 | 4249 | 2457 | 1.74 |
| Corn | Commercial | 38.5 | 2304 | 3969 | 2265 | 1.78 |
| | xylanase | | | | | |
| Wheat | Control | 38.4 | 2477 | 4056 | 2438 | 1.72 |
| Wheat | AclXyn5 | 38.3 | 2700 | 4215 | 2662 | 1.59 |
| Wheat | Commercial | 38.2 | 2456 | 4077 | 2418 | 1.71 |
| | xylanase | | | | | |

| | | | | | |
|---|---|---|---|---|---|
| SEM | 0.18 | 50.8 | 77.4 | 50.8 | 0.01 |
| Main effects, grains | | | | | |
| Corn | 38.3 | 2300b | 3967b | 2261b | 1.77a |
| Wheat | 38.3 | 2594a | 4146a | 2556a | 1.65b |
| SEM | 0.10 | 29.3 | 44.7 | 29.3 | 0.01 |
| Main effects, | | | | | |
| xylanases | | | | | |
| Control | 38.3 | 2314c | 3897b | 2276c | 1.76a |
| AclXyn5 | 38.3 | 2598a | 4232a | 2559a | 1.66b |
| Commercial xylanase | 38.3 | 2380b | 4023b | 2342b | 1.74a |
| SEM | 0.12 | 35.9 | 54.7 | 35.9 | 0.01 |
| Probabilities | | | | | |
| Grain | | <0.01 | 0.05 | <0.01 | <0.01 |
| Xylanase | | <0.01 | <0.01 | <0.01 | <0.01 |
| Grain and xylanase | | 0.23 | 0.09 | 0.23 | 0.03 |
| interaction | | | | | |

### Example 10

### 10.1 Materials and Methods

An experiment is conducted to evaluate the efficacy of AclXyn5 on energy and nutrient utilization/retention in broiler chickens fed corn/corn DDGS based diets and wheat/wheat bran based diets. An Animal Care and Use Committee approved all bird handling and collection procedures.

TABLE 10.1
Composition of the basal diets¹

| Ingredient | Corn based | Wheat based |
|---|---|---|
| Corn | 55.13 | — |
| Corn DDGS | 11.00 | — |
| Wheat | — | 56.90 |
| Wheat bran | — | 2.00 |
| Wheat middlings | — | 8.00 |
| Soybean meal | 28.94 | 26.19 |
| Soybean oil | 1.00 | 3.10 |
| L-Lysine•HCl | 0.43 | 0.38 |
| DL-Methionine | 0.27 | 0.24 |
| L-Threonine | 0.11 | 0.12 |
| Sodium bicarbonate | 0.20 | 0.20 |
| Salt | 0.22 | 0.22 |
| Limestone | 1.53 | 1.53 |
| Monocalcium phosphate | 0.56 | 0.51 |
| Vitamin-trace mineral premix² | 0.31 | 0.31 |
| Titanium dioxide | 0.30 | 0.30 |
| Calculated Provisions | | |
| Crude protein | 21.14 | 21.82 |
| ME (MJ/kg) | 11.51 | 11.51 |
| Calcium | 0.89 | 0.88 |
| Available P | 0.28 | 0.28 |
| Dig. lysine | 1.15 | 1.15 |
| Dig. methionine | 0.55 | 0.50 |

¹A commercial phytase from Danisco Animal Nutrition top dressed to supply 500 FTU/kg of final feed
²Supplied per kilogram of diet: antioxidant, 100 mg; biotin, 0.2 mg; calcium pantothenate, 12.8 mg; cholecalciferol, 60 μg; cyanocobalamin, 0.017 mg; folic acid, 5.2 mg; menadione, 4 mg; niacin, 35 mg; pyridoxine, 10 mg; trans-retinol, 3.33 mg; riboflavin, 12 mg; thiamine, 3.0 mg; dl-α-tocopheryl acetate, 60 mg; choline chloride, 638 mg; Co, 0.3 mg; Cu, 3.0 mg; Fe, 25 mg; I, 1 mg; Mn, 125 mg; Mo, 0.5 mg; Se, 200 μg; Zn, 60 mg.

Two basal diets, one based on corn/corn DDGS and another based on wheat/wheat bran/wheat middlings and diet are mixed (Table 10.1). From each of these basal diets, 3 experimental diets, all in mash form, are developed, using additives as shown in Table 10.2. To achieve the desired enzyme activities per kg of finished feed, enzyme pre-mixes (in powder form) were mixed into the feed at inclusion rate of 1000 g/tonne. A commercial phytase from Danisco Animal Nutrition top dressed to the both basal diets, so all the diets are supplied with 500 A commercial phytase from Danisco Animal Nutrition/kg of final feed. Titanium dioxide (0.3%) is added to all diets as an indigestible digesta marker.

TABLE 10.2
Treatments identification

| | | Phytase¹ | | |
|---|---|---|---|---|
| Diet | Treatment ID | (FTU/kg of feed) | Xylanase | |
| Control | 1 | 500 FTU | 0 | |
| Control + AclXyn5 | 2 | 500 FTU | 2500 | U/kg |
| Control + Commercial | 3 | 500 FTU | 50 | ppm |
| xylanase² | | | | |

¹Phytase from Danisco Animal Nutrition
²Econase ® XT from AB Vista

The study involves a cage trial, which is conducted to obtain excreta samples at day 21 for nutrient utilization measurements. Male broiler chicks (Ross 308) are obtained as day-olds from a commercial hatchery. The trial is conducted from day 13 to 21. Prior to the introduction to cages (from day 0 to 12), the birds are reared in floor pens and fed a commercial starter diet. On day 13, the chicks are individually weighed and allocated to 48 cages (six chicks per cage) so that the average bird weight per cage is similar. The 8 dietary treatments are then randomly assigned to six replicate cages in a 2×4 factorial treatment arrangements. The cages are housed in environmentally controlled rooms. The temperature is maintained at 26° C. on day 13 and then gradually reduced to 24° C. by day 21. The birds receive 20 hours of fluorescent illumination daily and, allowed free access to the diets and water throughout the 9-day experimental period. From day 17 to 20 post-hatch, feed intake and total excreta output are measured quantitatively per cage over four consecutive days. Excreta are pooled within a cage, mixed well using a blender and two representative samples per cage are taken. The samples are freeze-dried. Dried samples are ground to pass through a 0.5 mm sieve and stored in airtight plastic containers at −4° C. until chemical analyses. Samples of diets and excreta are analyzed for dry matter (DM), nitrogen (N), gross energy (GE), fat and neutral detergent fibre (NDF). Dry matter determination is carried out according to AOAC (1994) procedures. Nitrogen content is determined by the Dumas method (Sweeney, 1989) using a CNS-2000 carbon, N and sulphur analyser (LECO Corporation, St. Joseph, Mich.). Gross energy is determined using an adiabatic bomb calorimeter (Gallenkamp, London, UK), standardized with benzoic acid. Fibertec System M (Tecator, Höganäs, Sweden) is used for determination of NDF Fat content was determined following Soxhlet extraction procedure.

The AME values of the diets are calculated using the following formula, with appropriate corrections for differences in moisture content.

$$AME = \frac{( Feed intake \times {GE}_{diet} ) - ( Excreta output \times {GE}_{excreta} )}{Feed intake}$$

Apparent total tract retention coefficient of dry matter, fat, crude protein and NDF are determined as follows:

$$Retention coefficient = \frac{( Feed intake \times {Nutrient}_{diet} ) - ( Excreta output \times {Nutrient}_{excreta} )}{Feed intake \times {Nutrient}_{diet}}$$

Data are analyzed as a two-way factorial arrangement of treatments using the General Linear Models procedure of SAS (2004).

### 10.2 Results and Discussion

Birds fed AclXyn5 had significantly higher (P<0.05) apparent retention of dry matter, fat and crude protein than control fed birds (Table 6). This suggested that AclXyn5 is effective in unlocking energy in diverse fibrous cereal products by breaking down fibres. Indeed, birds fed AclXyn5 show increased fibre digestibility by a range of 1.7 to 6.5 percentage units relative to the control and a commercial xylanase (Table 10.3). Subsequently birds fed AclXyn5 extracted 71 extra (Table 10.3) energy compared to birds fed the control diet.

TABLE 10.3
Effect of new xylanase on nutrients and fibre retention (%)
and energy utilization (kcal/kg) in broiler chickens fed
corn-corn DDGS and wheat-wheat bran based diets

| | | | | | Neutral | |
|---|---|---|---|---|---|---|
| | | Dry | Crude | | detergent | |
| Treatments | | matter | protein | Fat | fibre | Energy |
| Grain | Xylanase | | | | | |
| Corn | Control | 70.3 | 66.0 | 83.7 | 31.7 | 3208 |
| Corn | AclXyn5 | 72.8 | 69.1 | 87.9 | 37.5 | 3304 |
| Corn | Commercial | 71.6 | 68.3 | 84.6 | 35.0 | 3260 |
| | xylanase | | | | | |
| Wheat | Control | 69.3 | 65.8 | 81.4 | 24.0 | 3204 |
| Wheat | AclXyn5 | 70.6 | 66.6 | 85.2 | 31.3 | 3250 |
| Wheat | Commercial | 70.4 | 67.0 | 85.1 | 30.4 | 3238 |
| | xylanase | | | | | |

| | | | | | |
|---|---|---|---|---|---|
| SEM | 0.39 | 0.59 | 0.97 | 1.07 | 17.7 |
| Main effects, grains | | | | | |
| Corn | 71.6a | 68.0a | 85.4 | 35.1a | 3257 |
| Wheat | 70.4b | 66.6b | 83.9 | 28.5b | 3231 |
| SEM | 0.22 | 0.34 | 0.56 | 0.62 | 9.93 |
| Main effects, | | | | | |
| xylanases | | | | | |
| Control | 69.8b | 65.9b | 82.6c | 27.9b | 3206b |
| AclXyn5 | 71.7a | 67.8a | 86.6ab | 34.4a | 3277a |
| Commercial xylanase 2 | 71.0a | 67.6a | 84.9b | 32.7b | 3250a |
| SEM | 0.27 | 0.42 | 0.69 | 0.76 | 12.3 |
| Probabilities | | | | | |
| Grain | <0.01 | 0.01 | 0.07 | <0.01 | 0.07 |
| Xylanase | <0.01 | 0.01 | <0.01 | <0.01 | <0.01 |
| Grain and xylanase | 0.28 | 0.18 | 0.21 | 0.38 | 0.34 |
| interaction | | | | | |

### Example 11

AclXyn5 in Combination with Protease

The effect of AclXyn5 in combination with protease was investigated on the solubilization of pentosan and protein from prepared insoluble DDGS

### Materials and Methods

### Enzyme Samples

The xylanase used in this study is a GH11 xylanase from Aspergillus clavatus (designated AclXyn 5) expressed in Trichoderma reesei, wherein the xylanase was used in purified form—this enzyme may be referred to herein as AclXyn5.

The protease used in this study is the Multifect P-3000 product (available from DuPont Industrial Biosciences). The preparation of protease was performed just prior to loadings.

Proper amount of stock solution was diluted in cooled MQ-water and mixed while kept on ice. One protease unit (U) was defined as the release of 1.0 μg of phenolic compound (expressed as tyrosine equivalents) from a casein substrate per minute.

### Substrate Preparation

For the preparation of insoluble DDGS substrate, removal of soluble non-starch polysaccharides (S-NSP) was performed according to Bach Knudsen (Bach Knudsen, K. E., Carbohydrate and lignin contents of plant materials used in animal feeding. Animal Feed Science and Technology 1997, 67, 319-338); Milled DDGS (<212 μm) and acetate/CaCl₂-buffer (0.1 M/20 mM, pH 5.0) was added together with thermostable α-amylase (E-BLAAM 53.7 U/mg, Megazyme International) and incubated for 1 h at 100° C. with frequent mixing.

Complete degradation of starch was done by incubation with amyloglucosidase (E-AMGDF 36 U/mg, Megazyme International) for 2 h at 60° C. After removal of the starch, the S-NSP was extracted by a phosphate buffer (0.2 M, pH 7.0) and placed at 100° C. for 1 h, followed by centrifugation. The pellet was then thoroughly washed with phosphate buffer, ethanol (85% v/v), and finally acetone, with centrifugation and discard of supernatant in between washes. The sample was placed at room temperature until completely dry.

### Procedure

FveXyn4 alone or in combination with protease was investigated on the solubilization of pentosan and protein of prepared insoluble DDGS. 87.5 mg of the prepared insoluble DDGS substrate was weighed into 1.5 ml eppendorf tubes and mixed with citrate buffer (25 mM, pH 6), xylanase (217 mg/kg substrate) and protease (8.6×10⁵ U/kg substrate) to a final reaction volume of 1.0 ml. The incubations were carried out at 4 h, 39° C. and 1300 rpm by use of Eppendorf ThermoMixer incubator (Eppendorf). After incubation, samples were filtered and analyzed for soluble pentosan and -protein content, as described below. Reactions were performed in duplicates.

### Protein Quantification

Soluble protein was quantified using the BCA (bicinchoninic acid) Protein Assay Kit from Pierce. The samples were prepared in microtiter plates (25 μl/well) and incubated with 200 μl premixed assay reagent for 30 minutes at 37° C., 1100 rpm. The absorbance was measured spectrophotometrically at 562 nm against a 0-2000 μg/ml Bovine Serum Albumin (BSA) standard, as described in the manual. Values were corrected for the amount of added enzymes.

### Quantification of C5 Sugars (Pentosans)

The total amount of pentoses brought into solution was measured using the method of Rouau and Surget (1994, A rapid semi-automated method of the determination of total and water-extractable pentosan in wheat flours. Carbohydrate Polymers, 24, 123-32) with a continuous flow injection apparatus (FIG. 7). The supernatants were treated with acid to hydrolyse polysaccharides to monosugars. Phloroglucinol (1, 3, 5-trihydroxybenzen) was added for reaction with monopentoses and monohexoses, which forms a coloured complex.

By measuring the difference in absorbance at 550 nm compared to 510 nm, the amount of pentoses in the solution was calculated using a standard curve. Unlike the pentose-phioroglucinol complex, the absorbance of the hexose-phioroglucinol complex is constant at these wavelengths. Glucose was added to the phloroglucinol solution to create a constant glucose signal and further ensure no interference from hexose sugars.

### Statistical Analysis

A one-way ANOVA was applied on the experimental data for comparison of treatments on both the solubilization of pentosan and protein, with pairwise comparisons performed by Holm-Sidak method, using SigmaPlot 12.0 (SyStat Software Inc.). Overall significance level at P=0.05.

### Results and Discussion

Pentosan and protein solubilization was measured by incubation of insoluble corn DDGS with xylanase and protease alone and in combination.

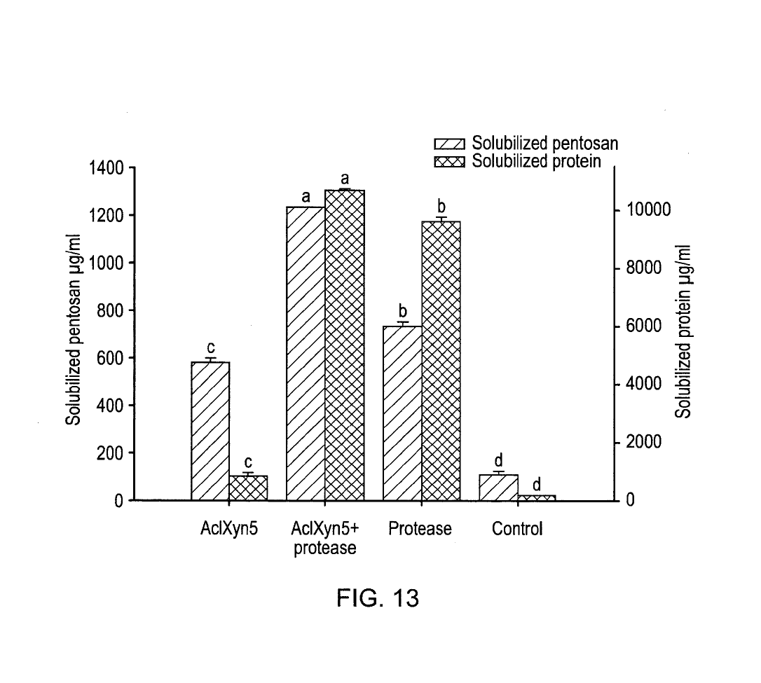

The results are shown in FIG. 13 (insoluble corn DDGS).

FIG. 13 shows the effect of the xylanase and protease treatments alone and in combination on the solubilization of pentosan and protein from insoluble corn DDGS. Letters a-d are significant different according to on-way ANOVA and Holm-Sidak comparisons with overall significance level at P=0.05. Error bars indicate S.D.

When compared to the effects of xylanase treatment by itself, the combination of xylanase and protease further increased the solubillization of protein from corn DDGS. More interestingly, addition of protease also significantly increased the solubilization of pentosan from corn DDGS, indicating a synergistic effect where addition of protease increase the accessibility of the xylanase towards the substrate by opening up the feed matrix structure through protein degradation. Furthermore, xylanase by itself and in combination with protease also increase the solubilization of protein as compared to control and protease alone, respectively. This further supports the theory of a synergistic effect between xylanase and protease.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method of preparing a corn based product comprising contacting a plant composition comprising corn or a corn by-product or a combination thereof with:
(a) a xylanase comprising a polypeptide as set forth in SEQ ID No. 8 or SEQ ID No. 7 or SEQ ID No. 6; or a variant, homologue or derivative thereof having xylanase activity having at least 85% identity with SEQ ID No. 8 or SEQ ID No. 7 or SEQ ID No. 6; or
(b) a xylanase encoded by a nucleotide sequence shown herein as SEQ ID No. 3, SEQ ID No. 2 or SEQ ID No. 1, or a nucleotide sequence which can hybridize to the complement of SEQ ID No. 3, SEQ ID No. 2 or SEQ ID No. 1 under high stringency conditions or
(c) a olypetide having xylanase activity encoded by a nucleotide sequence which has at least 80% identity with SEQ ID No. 3, SEQ ID No. 2 or SEQ ID No. 1.

2. The method of claim 1, wherein the corn by-product is corn gluten meal or corn Distillers Dried Grain Solubles (DDGS).

3. The method of claim 1, wherein the the plant composition is further contacted with one or more enzymes selected from the group consisting of a protease, a bacillolysin (E.C. 3.4.24.28), an alkaline serine protease (E.C. 3.4.21.x), a keratinase (E.C. 3.4.x.x)), and an amylase.

4. The method of claim 1, wherein the xylanase is used in combination with an amylase and a protease.

5-16. (canceled)

17. A method for increasing (i) weight gain; and/or (ii) digestibility; and/or (iii) improving feed efficiency in a subject comprising administering:
(a) a feed additive composition comprising a xylanase comprising a polypeptide as set forth in SEQ ID No. 8 or SEQ ID No. 7 or SEQ ID No. 6; or a variant, fragment, homologue or derivative thereof having xylanase activity having at least 85% identity with SEQ ID No. 8 or SEQ ID No. 7 or SEQ ID No. 6; or
(b) a xylanase encoded by a nucleotide sequence shown herein as SEQ ID No. 3, SEQ ID No. 2 or SEQ ID No. 1, or a nucleotide sequence which can hybridize to the complement of SEQ ID No. 3, SEQ ID No. 2 or SEQ ID No. 1 under high stringency conditions; or
(c) a polypeptide having xylanase activity encoded by a nucleotide sequence which has at least 80% identity with SEQ ID No. 3, SEQ ID No. 2 or SEQ ID No. 1
to the subject, thereby increasing (i) weight gain; and/or (ii) digestibility; and/or (iii) improving feed efficiency in the subject.

18-22. (canceled)

23. The method of claim 3, wherein the protease is a subtilisin (E.C. 3.4.21.62).

24. The method of claim 3, wherein the amylase is one or more of an α-amylase (E.C. 3.2.1.1), a G4-forming amylase (E.C. 3.2.1.60), a β-amylase (E.C. 3.2.1.2) and/or a γ-amylase (E.C. 3.2.1.3).

25. The method of claim 3, wherein the xylanase or the polypeptide having xylanase activity is derived from a fungus.

26. The method of claim 17, further comprising simultaneously or sequentially administering a plant composition comprising corn or a corn by product to the subject.

27. The method of claim 26, wherein the corn by-product is corn gluten meal or corn Distillers Dried Grain Solubles (DDGS).

28. The method of claim 17, wherein improving digestibility comprises improving nutrient digestibility.

29. The method of claim 17, further comprising simultaneously or sequentially administering one or more enzymes selected from the group consisting of a protease, a bacillolysin (E.C. 3.4.24.28), an alkaline serine protease (E.C. 3.4.21.x), a keratinase (E.C. 3.4.x.x)), and an amylase.

30. The method of claim 29, wherein the protease is a subtilisin (E.C. 3.4.21.62).

31. The method of claim 29, wherein the amylase is one or more of an α-amylase (E.C. 3.2.1.1), a G4-forming amylase (E.C. 3.2.1.60), a j3-amylase (E.C. 3.2.1.2) and/or a γ-amylase (E.C. 3.2.1.3).

32. The method of claim 17, wherein the subject is an animal selected from the group consisting of cattle, pigs, sheep, goats, poultry, deer, elk, reindeer, buffalo, bison, antelope, camels, kangaroos, horses, fish, cats, dogs, guinea pigs, and rodents.

33. The method of claim 17, wherein the xylanase or the polypeptide having xylanase activity is derived from a fungus.
